(19) United States
(12) Patent Application Publication
(10) Pub. No.: US 2026/0000899 A1
(43) Pub. Date: Jan. 1, 2026

(54) METHOD AND APPARATUS FOR CARDIAC EVENT SIGNAL SENSING

(71) Applicant: Medtronic, Inc., Minneapolis, MN (US)

(72) Inventors: Yanina GRINBERG, Plymouth, MN (US); Saul E. GREENHUT, Denver, CO (US); Robert A. BETZOLD, Fridley, MN (US); Troy E. JACKSON, Rogers, MN (US); Vincent P. GANION, Blaine, MN (US); Michael L. HUDZIAK, Stillwater, MN (US); Todd J. SHELDON, North Oaks, MN (US)

(21) Appl. No.: 18/992,965

(22) PCT Filed: Jul. 11, 2023
(86) PCT No.: PCT/US2023/069945

§ 371 Date: Jan. 9, 2025

Related U.S. Application Data
(60) Provisional application No. 63/368,241, filed on Jul. 12, 2022.

(51) Int. Cl. A61N 1/365; A61N 1/37

(52) U.S. Cl. CPC A61N 1/365; A61N 1/37

(57) ABSTRACT

A medical device is configured to sense a first cardiac signal and a second cardiac signal and determine a quantitative relationship of a first feature of the first cardiac signal and a second feature of the second cardiac signal. The medical device is configured to confirm a sensed cardiac event signal and/or reject an oversensed signal based on the quantitative relationship.

### REFERENCE TO RELATED APPLICATIONS

This application claims the benefit of provisional U.S. Patent Application No. 63/368,241, filed on Jul. 12, 2022, the entire content of which is incorporated herein by reference.

### TECHNICAL FIELD

This disclosure relates to a medical device configured to sense cardiac event signals and discriminate between a sensed cardiac event signal and a cross-chamber oversensed cardiac event signal.

### BACKGROUND

During normal sinus rhythm (NSR), the heartbeat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (AV) node. The AV node responds by propagating a ventricular depolarization signal through the bundle of His of the ventricular septum and thereafter to the bundle branches and the Purkinje muscle fibers of the right and left ventricles, sometimes referred to as the “His-Purkinje system.”

Patients with a conduction system abnormality, e.g., SA node dysfunction or poor AV node conduction, bundle branch block, or other conduction abnormalities, may receive a pacemaker to restore a more normal heart rhythm. A single chamber pacemaker coupled to a transvenous lead carrying electrodes positioned in the right atrium may provide atrial pacing to treat a patient having SA node dysfunction. When the AV node is functioning normally, single chamber atrial pacing may sufficiently correct the heart rhythm. The pacing-evoked atrial depolarizations may be conducted normally to the ventricles via the AV node and the His-Purkinje system maintaining normal AV synchrony. Some patients, however, may experience conduction abnormalities of the AV node, e.g., partial or complete AV block. AV block may be intermittent and may evolve over time. In the presence of high-degree AV block, atrial depolarizations may not be conducted to the ventricles on every atrial cycle or may be conducted but at a prolonged AV conduction time resulting in poor AV synchrony in the native heart rhythm. In this case, the patient may require a single chamber ventricular pacemaker or a dual chamber pacemaker.

A dual chamber pacemaker may be implanted in some patients to pace both the atrial and ventricular chambers and thereby maintain AV synchrony. The dual chamber pacemaker may be coupled to a transvenous atrial lead and a transvenous ventricular lead, for placing electrodes for sensing and pacing in both the atrial and ventricular chambers. The pacemaker itself can be implanted in a subcutaneous pocket with the transvenous leads tunneled to the subcutaneous pocket.

Intracardiac pacemakers have been introduced or proposed for implantation entirely within a patient's heart eliminating the need for transvenous leads. For example, an intracardiac pacemaker may provide sensing and pacing from within a heart chamber of a patient having a conduction abnormality to promote a more normal heart rhythm.

### SUMMARY

The techniques of this disclosure generally relate to a medical device configured to sense cardiac event signals attendant to myocardial depolarizations. The medical device may receive a cardiac electrical signal from electrodes implanted in or on a heart chamber and sense the cardiac event signals from the cardiac electrical signal. The medical device may be a pacemaker configured to sense P-waves attendant to atrial depolarizations and/or R-waves attendant to ventricular depolarizations. The medical device may be capable of generating cardiac pacing pulses. The timing of generated cardiac pacing pulses may be controlled by the medical device based on the sensed cardiac event signals.

A medical device operating according to the techniques disclosed herein may sense a cardiac event signal from the received cardiac electrical signal and determine if the sensed cardiac event signal corresponds to a first heart chamber (e.g., atrial or ventricular) or a second heart chamber (e.g., ventricular or atrial). A first cardiac event signal may be confirmed as arising from the first heart chamber if a second cardiac event signal is not sensed within a confirmation delay time interval. The first cardiac event signal may be confirmed as arising from the first heart chamber if a second cardiac event signal is sensed during the confirmation delay time interval and a ratio, difference or other quantitative relationship of a feature of the first cardiac event signal and a feature of the second cardiac event signal meets a confirmation threshold for confirming the first cardiac event signal. The first cardiac event signal may be confirmed as arising from the second heart chamber if the second cardiac event signal is sensed during the confirmation delay time interval and a quantitative relationship of a feature of the first cardiac event signal and the second cardiac event signal does not meet the confirmation threshold required for confirming the first cardiac event signal.

In one example, the disclosure provides a medical device including sensing circuitry configured to sense a first cardiac signal and a second cardiac signal. The medical device includes a control circuit configured to receive a first event signal of the first cardiac signal from the sensing circuitry and receive a second event signal of the second cardiac signal from the sensing circuitry. The control circuit can be configured to determine a quantitative relationship between a first feature of the first cardiac signal and a second feature of the second cardiac signal and confirm a sensed cardiac event signal based on the quantitative relationship as one of the first event signal or the second event signal.

In another example, the disclosure provides a method including sensing a first cardiac signal and a second cardiac signal, receiving a first event signal of the first cardiac signal, receiving a second event signal of the second cardiac signal, determining a quantitative relationship of a first feature of the first cardiac signal and a second feature of the second cardiac signal and confirming a sensed cardiac event signal based on the quantitative relationship as one of the first event signal or the second event signal.

In another example, the disclosure provides a non-transitory, computer-readable storage medium comprising a set of instructions which, when executed by a control circuit of a medical device, cause the medical device to sense a first cardiac signal and a second cardiac signal, receive a first event signal of the first cardiac signal, receive a second event signal of the second cardiac signal, determine a quantitative relationship of a first feature of the first cardiac signal and a second feature of the second cardiac signal, and confirm a sensed cardiac event signal based on the quantitative relationship as one of the first event signal or the second event signal.

Further disclosed herein is the subject matter of the following clauses:

Clause 1. A medical device comprising sensing circuitry configured to sense a first cardiac signal and a second cardiac signal and a control circuit configured to receive a first event signal of the first cardiac signal from the sensing circuitry and receive a second event signal of the second cardiac signal from the sensing circuitry. The control circuit can be further configured to determine a quantitative relationship between a first feature of the first cardiac signal and a second feature of the second cardiac signal and confirm a sensed cardiac event signal based on the quantitative relationship as one of the first event signal or the second event signal.

Clause 2. The medical device of clause 1 wherein the control circuit is further configured to schedule a cardiac pacing pulse at one of: a first pacing interval in response to confirming the sensed cardiac event signal as the first cardiac event signal or a second pacing interval different than the first pacing interval in response to confirming the sensed cardiac event signal as the second cardiac event signal.

Clause 3. The medical device of clause 2 further comprising a pulse generator configured to deliver the cardiac pacing pulse scheduled at one of the first pacing interval or the second pacing interval.

Clause 4. The medical device of any of clauses 1-3 wherein the sensing circuitry is further configured to sense a first cardiac event signal in response to the first cardiac signal crossing a first sensing threshold produce the first event signal received by the control circuit, sense a second cardiac event signal in response to the second cardiac electrical signal crossing a second sensing threshold, and produce the second event signal received by the control circuit.

Clause 5. The medical device of any of clauses 1-4 wherein the control circuit is further configured to confirm the sensed cardiac event signal based on the quantitative relationship by: comparing the quantitative relationship to a first confirmation threshold, confirm the sensed cardiac event signal as being the first cardiac event signal in response to the quantitative relationship meeting the first confirmation threshold or confirm the sensed cardiac event signal as being the second cardiac event signal in response to the quantitative relationship not meeting the first confirmation threshold.

Clause 6. The medical device of any of clauses 1-5 wherein the control circuit is further configured to receive the first event signal as an atrial event signal, receive the second event signal as a ventricular event signal, and compare the quantitative relationship to a P-wave confirmation threshold when the first event signal is the atrial event signal. The control circuit can be further configured to confirm the sensed cardiac event signal based on the quantitative relationship by confirming the sensed cardiac event signal as being the atrial event signal in response to the quantitative relationship being less than the P-wave confirmation threshold or confirming the sensed cardiac event signal as being a ventricular event signal in response to the quantitative relationship being greater than the P-wave confirmation threshold.

Clause 7. The medical device of any of clauses 1-5 wherein the control circuit is further configured to receive the first event signal as a ventricular event signal, receive the second event signal as an atrial event signal and compare the quantitative relationship to an R-wave confirmation threshold when the first event signal is the ventricular event signal. The control circuit can be further configured to confirm the sensed cardiac event signal based on the quantitative relationship by confirming the sensed cardiac event signal as being the ventricular event signal in response to the quantitative relationship being greater than the R-wave confirmation threshold or confirming the sensed cardiac event signal as being the atrial event signal in response to the quantitative relationship being less than the R-wave confirmation threshold.

Clause 8. The medical device of any of clauses 1-5 wherein the control circuit is further configured to confirm the sensed cardiac event signal based on the quantitative relationship by comparing the quantitative relationship to a P-wave confirmation threshold and to an R-wave confirmation threshold and confirming the sensed cardiac event signal as being one of an atrial event signal in response to the quantitative relationship being closer to the P-wave confirmation threshold than the R-wave confirmation threshold or a ventricular event signal in response to the quantitative relationship being closer to the R-wave confirmation threshold than to the P-wave confirmation threshold.

Clause 9. The medical device of any of clauses 1-8 wherein the control circuit is further configured to determine the quantitative relationship by determining the first feature as a maximum peak amplitude of the first cardiac signal and the second feature as a maximum peak amplitude of the second cardiac signal.

Clause 10. The medical device of any of clauses 1-9 wherein the control circuit is further configured to start one or more blanking period(s) and/or one or more refractory period(s) based on the confirmed sensed cardiac event signal.

Clause 11. The medical device of any of clauses 1-10 wherein the control circuit is further configured to adjust at least one of: a first low pass filter cutoff frequency applied to the first cardiac signal by the sensing circuitry, a first high pass filter cutoff frequency applied to the first cardiac signal by the sensing circuitry, a second low pass filter cutoff frequency applied to the second cardiac signal by the sensing circuitry; and/or a second high pass filter cutoff frequency applied to the second cardiac signal by the sensing circuitry.

Clause 12. The medical device of clause 11 wherein the control circuit is further configured to determine a first quantitative relationship of the first feature of the first cardiac signal and the second feature of the second cardiac signal in response to receiving the first event signal from the sensing circuitry without receiving the second event signal within a confirmation delay time from the first event signal. The control circuit may determine a second quantitative relationship of the first feature of the first cardiac signal and the second feature of the second cardiac signal in response to receiving the second event signal from the sensing circuitry without receiving the first event signal within a confirmation delay time from the first event signal. The control circuit may determine a difference between the first quantitative relationship and the second quantitative relationship and adjust at least one of the first low pass filter cutoff frequency, the first high pass filter cutoff frequency, the second low pass filter cutoff frequency, and/or the second high pass filter cutoff frequency to maximize the difference between the first quantitative relationship and the second quantitative relationship.

Clause 13. The medical device of any of clauses 1-12 wherein the control circuit is further configured to determine that the second cardiac event signal is received within a confirmation delay time from the first cardiac event signal and determine the quantitative relationship in response to the second cardiac event signal being received within the confirmation delay time.

Clause 14. The medical device of clause 13 wherein the control circuit is further configured to schedule a pacing pulse by starting a pacing interval according to which of the first cardiac event signal or the second cardiac event signal is confirmed and adjusting the pacing interval by one of the confirmation delay time or a time since the second cardiac event signal.

Clause 15. The medical device of any of clauses 1-14 wherein the sensing circuitry includes a first sensing circuit configured to receive the first cardiac signal and a second sensing circuit configured to receive the second cardiac signal.

Clause 16. The medical device of any of clauses 1-15 wherein the control circuit is further configured to determine the quantitative relationship as a ratio of the first feature and the second feature.

Clause 17. The medical device of any of clauses 1-15 wherein the control circuit is further configured to determine the quantitative relationship as one of: a ratio of the first feature and the second feature; a difference between the first feature and the second feature, a sum of the first feature and the second feature, or a product of the first feature and the second feature.

Clause 18. The medical device of any of clauses 1-17 wherein the control circuit is further configured to set a first confirmation threshold by receiving the first event signal from the sensing circuitry without receiving the second event signal within a confirmation delay time from the first event signal, determining a first reference quantitative relationship of the first feature of the first cardiac signal and the second feature of the second cardiac signal when the first event signal is received from the sensing circuitry without receiving the second event signal within the confirmation delay time from the first event signal and setting the first confirmation threshold based on the first reference quantitative relationship. The control circuit may be further configured to confirm the sensed cardiac event signal as one of the first event signal or the second event signal based on a comparison of the quantitative relationship to the first confirmation threshold.

Clause 19. The medical device of clause 18 wherein the control circuit is further configured to set a second confirmation threshold by receiving the second event signal from the sensing circuitry without receiving the first event signal within a confirmation delay time from the second event signal, determining a second reference quantitative relationship of the first feature of the first cardiac signal and the second feature of the second cardiac signal when the first event signal is received from the sensing circuitry without receiving the second event signal within the confirmation delay time from the first event signal and setting the second confirmation threshold based on the second reference quantitative relationship. The control circuit may confirm the sensed cardiac event signal as one of the first event signal or the second event signal based on a comparison of the quantitative relationship to at least one of the first confirmation threshold or the second confirmation threshold.

Clause 20. The medical device of any of clauses 1-17, wherein the control circuit is further configured to confirm the sensed cardiac event signal as one of the first event signal or the second event signal based on a comparison of the quantitative relationship to a confirmation threshold. The control circuit may be further configured to adjust the confirmation threshold based on the first cardiac signal and the second cardiac signal.

Clause 21. The medical device of any of clauses 1-20 further comprising a pulse generator configured to generate a pacing pulse. The control circuit being further configured to start a pacing escape interval to schedule a pending pacing pulse, start a confirmation delay in response to receiving the first event signal, determine that the pacing escape interval expires during the confirmation delay and delay the pending pacing pulse in response to the determining that the pacing escape interval expires during the confirmation delay. The control circuit further configured to confirm the sensed cardiac event signal as one of the first event signal or the second event signal based on the quantitative relationship and control the pulse generator to deliver or withhold the delayed pending pacing pulse according to whether the sensed cardiac event signal is confirmed as the first event signal or the second event signal.

Clause 22. The medical device of any of clauses 1-20 further including a pulse generator configured to generate a pacing pulse. The control circuit can be further configured to start a pacing escape interval to schedule a pending pacing pulse and, after starting the pacing escape interval, receive a next event signal from the sensing circuitry after confirming the sensed cardiac event signal. The control circuit may start a confirmation delay in response to receiving the next event signal. The control circuit may determine that the pacing escape interval expires before an expiration of the confirmation delay and before another event signal is received from the sensing circuitry. In response to the pacing escape interval expiring during the confirmation delay before another event signal is received from the sensing circuitry, the control circuit may control the pulse generator to deliver the scheduled pending pacing pulse upon expiration of the pacing escape interval.

Clause 23. The medical device of clause 22 wherein the control circuit is further configured start the pacing escape interval in response to the confirmed sensed cardiac event signal.

Clause 24. The medical device of clause 22 wherein the pulse generator is further configured to deliver a first pacing pulse before receiving the next event signal, and the control circuit is further configured to start the pacing escape interval to schedule the pending pacing pulse in response to the pulse generator delivering the first pacing pulse.

Clause 25. The medical device of any of clauses 21-24 further comprising a pulse generator configured to deliver a preceding pacing pulse prior to the control circuit receiving the first event signal. The control circuit being further configured to start a polarization delay in response to the pulse generator delivering the preceding pacing pulse. The control circuit may be further configured to detect an expiration of the polarization delay and start the confirmation delay in response to the first event signal when the first event signal is received after the polarization delay expires.

Clause 26. A method comprising sensing a first cardiac signal and a second cardiac signal, receiving a first event signal of the first cardiac signal, receiving a second event signal of the second cardiac signal, determining a quantitative relationship of a first feature of the first cardiac signal and a second feature of the second cardiac signal and confirming a sensed cardiac event signal as one of the first event signal or the second event signal based on the quantitative relationship.

Clause 27. The method of clause 26 further comprising scheduling a cardiac pacing pulse at one of a first pacing interval in response to confirming the sensed cardiac event signal as the first cardiac event signal or a second pacing interval different than the first pacing interval in response to confirming the sensed cardiac event signal as the second cardiac event signal.

Clause 28. The method of clause 27 further comprising delivering the cardiac pacing pulse scheduled at one of the first pacing interval or the second pacing interval.

Clause 29. The method of any of clauses 26-28 further comprising sensing a first cardiac event signal in response to the first cardiac signal crossing a first sensing threshold, producing the first event signal in response to sensing the first cardiac event signal, sensing a second cardiac event signal in response to the second cardiac electrical signal crossing a second sensing threshold, and producing the second event signal in response to sensing the second cardiac event signal.

Clause 30. The method of any of clauses 26-29 wherein confirming the sensed cardiac event signal based on the quantitative relationship comprises comparing the quantitative relationship to a first confirmation threshold, confirming the sensed cardiac event signal as being the first event signal in response to the quantitative relationship meeting the first confirmation threshold or confirming the sensed cardiac event signal as being the second event signal in response to the quantitative relationship not meeting the first confirmation threshold.

Clause 31. The method of any of clauses 26-30 further comprising receiving the first event signal as an atrial event signal, receiving the second event signal as a ventricular event signal, comparing the quantitative relationship to a P-wave confirmation threshold when the first event signal is the atrial event signal, and confirming the sensed cardiac event signal based on the quantitative relationship by confirming the sensed cardiac event signal as being the atrial event signal in response to the quantitative relationship being less than the P-wave confirmation threshold or confirming the sensed cardiac event signal as being the ventricular event signal in response to the quantitative relationship being greater than the P-wave confirmation threshold.

Clause 32. The method of any of clauses 26-30 further comprising receiving the first event signal as a ventricular event signal, receiving the second event signal as an atrial event signal, comparing the quantitative relationship to an R-wave confirmation threshold when the first cardiac event signal is a ventricular event signal, and confirming the sensed cardiac event signal based on the quantitative relationship by confirming the sensed cardiac event signal as being the ventricular event signal in response to the quantitative relationship being greater than the R-wave confirmation threshold or confirming the sensed cardiac event signal as being the atrial event signal in response to the quantitative relationship being less than the R-wave confirmation threshold.

Clause 33. The method of any of clauses 26-30, wherein confirming the sensed cardiac event signal based on the quantitative relationship comprises comparing the quantitative relationship to a P-wave confirmation threshold and to an R-wave confirmation threshold and confirming the sensed cardiac event signal as being one of: an atrial event signal in response to the quantitative relationship being closer to the P-wave confirmation threshold than the R-wave confirmation threshold or a ventricular event signal in response to the quantitative relationship being closer to the R-wave confirmation threshold than the P-wave confirmation threshold.

Clause 34. The method of any of clauses 26-33 further comprising determining the quantitative relationship by determining the first feature as a maximum peak amplitude of the first cardiac signal and the second feature as a maximum peak amplitude of the second cardiac signal.

Clause 35. The method of any of clauses 26-34 further comprising starting one or more blanking period(s) and/or one or more refractory period(s) based on the confirmed sensed cardiac event signal.

Clause 36. The method of any of clauses 26-35 further comprising adjusting at least one of: a first low pass filter cutoff frequency applied to the first cardiac signal; a first high pass filter cutoff frequency applied to the first cardiac signal; a second low pass filter cutoff frequency applied to the second cardiac signal; and/or a second high pass filter cutoff frequency applied to the second cardiac signal.

Clause 37. The method of clause 36 further comprising determining a first quantitative relationship of the first feature of the first cardiac signal and the second feature of the second cardiac signal in response to receiving the first event signal from the sensing circuitry without receiving the second event signal within a confirmation delay time from the first event signal, determining a second quantitative relationship of the first feature of the first cardiac signal and the second feature of the second cardiac signal in response to receiving the second event signal from the sensing circuitry without receiving the first event signal within a confirmation delay time from the first event signal. The method may further include determining a difference between the first quantitative relationship and the second quantitative relationship and adjusting at least one of the first low pass filter cutoff frequency, the first high pass filter cutoff frequency, the second low pass filter cutoff frequency, and/or the second high pass filter cutoff frequency to maximize the difference between the first quantitative relationship and the second quantitative relationship.

Clause 38. The method of any of clauses 26-37 further comprising determining that the second cardiac event signal is received within a confirmation delay time from the first cardiac event signal and determining the quantitative relationship in response to the second cardiac event signal being received within the confirmation delay time.

Clause 39. The method of clause 38 wherein the control circuit is further configured to schedule a pacing pulse by starting a pacing interval according to which of the first cardiac event signal or the second cardiac event signal is confirmed and adjusting the pacing interval by one of the confirmation delay time or a time since the second cardiac event signal.

Clause 40. The medical device of any of clauses 26-39 further comprising determining the quantitative relationship as a ratio of the first feature and the second feature.

Clause 41. The medical device of any of clauses 26-39 further comprising determining the quantitative relationship as one of: a ratio of the first feature and the second feature, a difference between the first feature and the second feature; a sum of the first feature and the second feature or a product of the first feature and the second feature.

Clause 42. The method of any of clauses 26-41 further comprising setting a first confirmation threshold by receiving the first event signal from the sensing circuitry without receiving the second event signal within a confirmation delay time from the first event signal, determining a first reference quantitative relationship of the first feature of the first cardiac signal and the second feature of the second cardiac signal when the first event signal is received from the sensing circuitry without receiving the second event signal within the confirmation delay time from the first event signal; and setting the first confirmation threshold based on the first reference quantitative relationship. The method may further include confirming the sensed cardiac event signal as one of the first event signal or the second event signal based on a comparison of the quantitative relationship to the first confirmation threshold.

Clause 43. The method of clause 42 further comprising setting a second confirmation threshold by receiving the second event signal from the sensing circuitry without receiving the first event signal within a confirmation delay time from the second event signal, determining a second reference quantitative relationship of the first feature of the first cardiac signal and the second feature of the second cardiac signal when the first event signal is received from the sensing circuitry without receiving the second event signal within the confirmation delay time from the first event signal, and setting the second confirmation threshold based on the second reference quantitative relationship. The method may include confirming the sensed cardiac event signal as one of the first event signal or the second event signal based on a comparison of the quantitative relationship to at least one of the first confirmation threshold or the second confirmation threshold.

Clause 44. The method of any of clauses 26-41 further comprising confirming the sensed cardiac event signal as one of the first event signal or the second event signal based on a comparison of the quantitative relationship to a confirmation threshold. The method may further include adjusting the confirmation threshold based on the first cardiac signal and the second cardiac signal.

Clause 45. The method of any of clauses 26-44 further comprising starting a pacing escape interval to schedule a pending pacing pulse, starting a confirmation delay in response to receiving the first event signal, determining that the pacing escape interval expires during the confirmation delay and delaying the pending pacing pulse in response to the determining that the pacing escape interval expires during the confirmation delay.

The method further including confirming the sensed cardiac event signal as one of the first event signal or the second event signal based on the quantitative relationship and controlling the pulse generator to deliver or withhold the delayed pending pacing pulse according to whether the sensed cardiac event signal is confirmed as the first event signal or the second event signal.

Clause 46. The method of any of clauses 26-44 further including starting a pacing escape interval to schedule a pending pacing pulse and, after starting the pacing escape interval, receiving a next event signal after confirming the sensed cardiac event signal. The method may further include starting a confirmation delay in response to receiving the next event signal, determining that the pacing escape interval expires before an expiration of the confirmation delay and before another event signal is received. In response to the pacing escape interval expiring during the confirmation delay before another event signal is received, the method may further include delivering the scheduled pending pacing pulse upon expiration of the pacing escape interval.

Clause 47. The method of clause 46 further including starting the pacing escape interval in response to the confirmed sensed cardiac event signal.

Clause 48. The method of clause 46 further including delivering a first pacing pulse before receiving the next event signal and starting the pacing escape interval to schedule the pending pacing pulse in response delivering the first pacing pulse.

Clause 49. The method of any of clauses 45-48 further including delivering a preceding pacing pulse prior to receiving the first event signal, starting a polarization delay in response to the pulse generator delivering the preceding pacing pulse, detecting an expiration of the polarization delay, and starting the confirmation delay in response to the first event signal when the first event signal is received after the polarization delay expires.

Clause 50. A non-transitory, computer-readable storage medium storing a set of instructions which, when executed by a control circuit of a medical device, cause the medical device to sense a first cardiac signal and a second cardiac signal, receive a first event signal of the first cardiac signal, receive a second event signal of the second cardiac signal and determine a quantitative relationship of a first feature of the first cardiac signal and a second feature of the second cardiac signal. The instructions may further cause the medical device to confirm a sensed cardiac event signal based on the quantitative relationship as one of the first event signal or the second event signal.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device (IMD) system that may be used to sense cardiac signals and provide cardiac pacing.

FIG. 2 is a conceptual diagram of the pacemaker shown in FIG. 1 according to one example.

FIG. 3 is a conceptual diagram of an example configuration of the pacemaker of FIG. 1 according to some examples.

FIG. 4 is a conceptual diagram of sensing circuitry that may be included in a pacemaker according to some examples.

FIG. 5 is a flow chart of a method that can be performed by a pacemaker for confirming a sensed cardiac event signal and identifying a cross-chamber oversensed (CCOS) event signal.

FIG. 6 is a timing diagram of an atrial electrogram (EGM) signal, a ventricular EGM signal and a sensed event marker channel that depicts cardiac sensed event signals that may be confirmed as true events or identified as CCOS events by a pacemaker according to some examples.

FIG. 7 is a state diagram illustrating sensing operating states of a pacemaker according to some examples.

FIG. 8 is a diagram of an atrial EGM signal and a ventricular EGM signal that may be sensed by a pacemaker.

FIG. 9 is a flow chart of a method that may be performed by a pacemaker for establishing or updating P-wave and/or R-wave confirmation thresholds according to some examples.

FIG. 10 is a flow chart of a method for establishing bandpass filter cutoff frequencies for sensing an atrial EGM signal and a ventricular EGM signal according to some examples.

FIG. 11 is a flow chart of a method that may be performed by a pacemaker for confirming sensed cardiac event signals according to another example.

FIG. 12 is a flow chart of a method for sensing cardiac event signals by a medical device and providing a pacing and/or sensing response to a sensed cardiac event signal according to another example.

FIGS. 13-17 are diagrams of various examples of a pacing response that a medical device may perform when a pacing escape interval expires during a confirmation delay.

### DETAILED DESCRIPTION

In general, this disclosure describes a medical device and method for determining when a cardiac event signal arising from a heart chamber, e.g., atrial or ventricular, is falsely oversensed as being a cardiac event signal arising from a different heart chamber, e.g., ventricular or atrial. An oversensed cardiac event signal, which may be a true atrial P-wave oversensed as a false ventricular R-wave or a true ventricular R-wave oversensed as a false atrial P-wave, may be identified by processing circuitry of the medical device according to the techniques disclosed herein. Sensing of an atrial electrical event as a false R-wave or sensing of a ventricular electrical event as a false P-wave is referred to herein as “cross-chamber oversensing.” Cross-chamber oversensing (CCOS) may cause the medical device to incorrectly determine a patient's heart rhythm or heart rate. CCOS may interfere with the timing and control of cardiac electrical stimulation therapies delivered by the medical device. For example, CCOS may cause the medical device to withhold an electrical stimulation pulse, e.g., a cardiac pacing pulse or other therapeutic electrical stimulation pulse, when the electrical stimulation pulse is actually needed or deliver an electrical stimulation pulse when the electrical stimulation pulse is actually not needed. Accordingly, the techniques disclosed herein provide improvements in the fields of cardiac rhythm monitoring and cardiac therapy delivery by improving cardiac event signal sensing functions of a medical device.

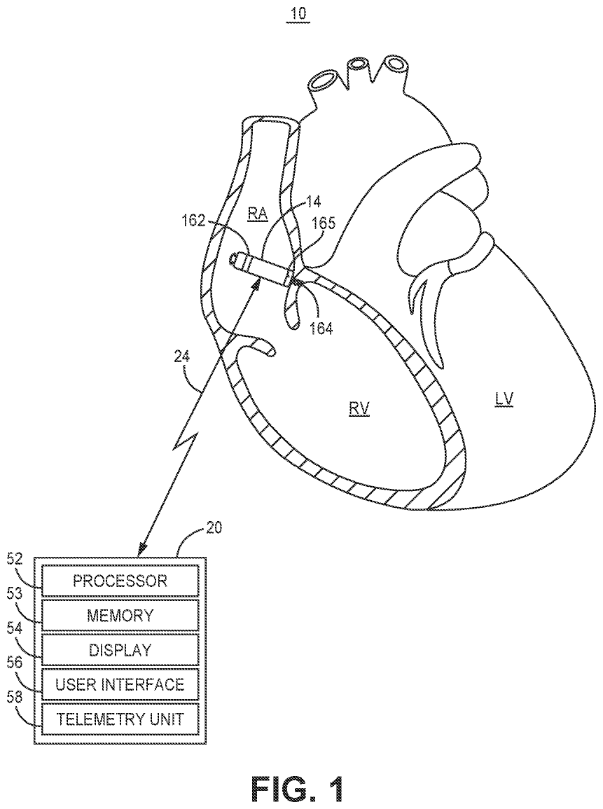

FIG. 1 is a conceptual diagram illustrating an implantable medical device (IMD) system 10 that may be used to sense cardiac signals and provide cardiac pacing. IMD system 10 is shown including a pacemaker 14, implanted within the right atrium (RA) of a patient's heart. In some examples, pacemaker 14 is a transcatheter, leadless pacemaker that can be implanted wholly within a heart chamber. Pacemaker 14 may be reduced in size compared to subcutaneously implanted pacemakers and may be generally cylindrical in shape to facilitate transvenous implantation via a delivery catheter. Pacemaker 14 may be a leadless pacemaker that includes electrodes carried on the pacemaker housing without requiring medical electrical leads extending from pacemaker 14 for sensing cardiac electrical signals and delivering cardiac pacing pulses.

Pacemaker 14 may be capable of sensing atrial and ventricular event signals, e.g., P-waves attendant to atrial depolarizations and R-waves attendant to ventricular depolarizations. Pacemaker 14 may be configured as a dual chamber pacemaker capable of sensing both atrial and ventricular event signals and delivering atrial pacing pulses and ventricular pacing pulses as needed based on the sensed atrial and/or ventricular event signals. In other examples, pacemaker 14 may be configured as a single chamber pacemaker capable of delivering only atrial pacing pulses or capable of delivering only ventricular pacing pulses but may still be capable of dual chamber sensing of both atrial and ventricular event signals. In still other examples, pacemaker 14 may be configured to sense and pace a single heart chamber, atrial or ventricular.

In the example shown, pacemaker 14 is implanted in the RA for providing ventricular pacing from an atrial location. Pacemaker 14 may be configured for delivering ventricular pacing pulses via the heart's native conduction system and/or ventricular myocardium from a RA approach. For example, the distal end of pacemaker 14 may be positioned at the inferior end of the interatrial septum, beneath the AV node and near the tricuspid valve annulus to position a tip electrode 164 for advancement into the interatrial septum toward the His bundle of the native His-Purkinje conduction system. A second electrode, e.g., a ring electrode 162 or ring electrode 165, may be spaced proximally from the tip electrode 164 for use with the tip electrode 164 for bipolar pacing of the right and left ventricles via the His-Purkinje system and/or ventricular myocardium. Ventricular pacing pulses delivered by pacemaker 14 may capture at least a portion of the His bundle and/or ventricular myocardium for delivering ventricular pacing to the ventricles, e.g., the right ventricle (RV) and/or left ventricle (LV), from an atrial implant location of pacemaker 14. The techniques disclosed herein are not necessarily limited to a particular implant location of pacemaker 14, however, and may be practiced in a pacemaker implanted in a variety of operative locations for providing cardiac signal sensing of atrial and/or ventricular events and, at least in some examples, delivering cardiac pacing to at least one heart chamber.

Pacemaker 14 may be capable of bidirectional wireless communication with an external device 20 for programming sensing and pacing control parameters. Aspects of external device 20 may generally correspond to the external programming/monitoring unit disclosed in U.S. Pat. No. 5,507,782 (Kieval, et al.), hereby incorporated herein by reference in its entirety. External device 20 is often referred to as a “programmer” because it is typically used by a physician, technician, nurse, clinician or other qualified user for programming operating parameters in an implantable medical device. External device 20 may be located in a clinic, hospital or other medical facility. External device 20 may alternatively be embodied as a home monitor or a handheld device that may be used in a medical facility, in the patient's home, or another location. Operating parameters, including sensing and therapy delivery control parameters, may be programmed into pacemaker 14 by a user interacting with external device 20.

External device 20 may include a processor 52, memory 53, display unit 54, user interface 56 and telemetry unit 58. Processor 52 controls external device operations and processes data and signals received from pacemaker 12 or 14. Display unit 54 may generate a display, which may include a graphical user interface, of data and information relating to pacemaker functions to a user for reviewing pacemaker operation and programmed parameters. Display unit 54 may generate a display that includes cardiac signals and/or data derived therefrom, cardiac pacing timing markers, cardiac pacing history and/or other physiological data, patient data or device-related data that may be stored by pacemaker 14 and transmitted to external device 20 during an interrogation session. For example, pacemaker 14 may generate an output for transmission to external device 20 including pacing and sensing event histories, device operating parameters and device diagnostic data.

Transmitted data may include an episode of a cardiac electrical signal produced by pacemaker sensing circuitry including markers indicating pacing pulse delivery and sensed cardiac event signals, e.g., ventricular sensed event signals and/or atrial sensed event signals and any delivered atrial and/or ventricular pacing pulses. The display unit 54 may display a cardiac electrical signal episode with annotated sensed event signals, pacing pulse markers, and/or identified oversensed event signals.

User interface 56 may include a mouse, touch screen, keypad or the like to enable a user to interact with external device 20 to initiate a telemetry session with pacemaker 14 for retrieving data from and/or transmitting data to the pacemaker 14, including programmable parameters for controlling sensing and pacing functions. Telemetry unit 58 includes a transceiver and antenna configured for bidirectional communication with a telemetry circuit included in pacemaker 14 and is configured to operate in conjunction with processor 52 for sending and receiving data relating to pacemaker functions via communication link 24.

Telemetry unit 58 may establish a wireless bidirectional communication link 24 with pacemaker 14. Communication link 24 may be established using a radio frequency (RF) link such as BLUETOOTH®, Wi-Fi, Medical Implant Communication Service

(MICS) or other communication bandwidth. In some examples, external device 20 may include a programming head that is placed proximate pacemaker 14 to establish and maintain a communication link 24, and in other examples external device 20 and pacemaker 14 may be configured to communicate using a distance telemetry algorithm and circuitry that does not require the use of a programming head and does not require user intervention to maintain a communication link.

It is contemplated that external device 20 may be in wired or wireless connection to a communications network via a telemetry circuit that includes a transceiver and antenna or via a hardwired communication line for transferring data to a centralized database or computer to allow remote management of the patient. Remote patient management systems including a centralized patient database may enable a clinician to view data relating to sensing and pacing functions performed by pacemaker 14.

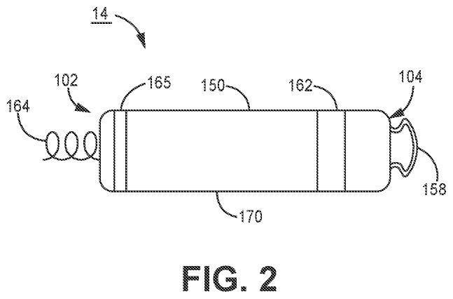

FIG. 2 is a conceptual diagram of the pacemaker 14 shown in FIG. 1 according to one example. Pacemaker 14 includes a housing 150 having a distal end 102 and a proximal end 104. The lateral sidewall 170 of housing 150 extending from distal end 102 to proximal end 104 may be generally cylindrical to facilitate transvenous delivery, e.g., via a catheter. Distal end 102 is referred to as “distal” in that it is expected to be the leading end as pacemaker 14 is advanced through a delivery tool, such as a catheter, and placed against a targeted pacing site. In other examples, housing 150 may have a generally prismatic shape. The housing 150 encloses the electronics and a power supply for sensing cardiac signals, producing pacing pulses and controlling therapy delivery and other functions of pacemaker 14 as described herein.

Pacemaker 14 is shown including electrodes 162, 164 and 165 spaced apart along the housing 150 of pacemaker 14 for sensing cardiac electrical signals and delivering pacing pulses. Electrode 164 is shown as a tip electrode extending from distal end 102 of housing 150. Electrodes 162 and 165 are shown as ring electrodes along the lateral sidewall 170 of housing 150. Electrodes 162 and 165 may be ring electrodes circumscribing the lateral sidewall 170, for example adjacent proximal end 104 and adjacent distal end 102, respectively.

Tip electrode 164 is shown as a screw-in helical electrode which may provide fixation of pacemaker 14 at an implant site as well as serving as a pacing and sensing electrode. Electrode 164 can be advanced from within the right atrial chamber to a ventricular pacing site, e.g., for delivering pacing to the His-Purkinje conduction system and/or for pacing of ventricular septal myocardial tissue.

Tip electrode 164 may serve as a cathode electrode with ring electrode 162 serving as a return anode for delivering ventricular pacing pulses. Tip electrode 164 and ring electrode 162 may be used as a bipolar pair for ventricular pacing and for receiving a ventricular electrical signal from which R-waves can be sensed by sensing circuitry enclosed by housing 150. Ring electrodes 162 and 165 may form a second anode and cathode pair for bipolar atrial pacing and sensing an atrial electrical signal from which P-waves can be sensed by the sensing circuitry enclosed by housing 150. Electrodes 162, 164 and 165 may be, without limitation, titanium, platinum, iridium or alloys thereof and may include a low polarizing coating, such as titanium nitride, iridium oxide, ruthenium oxide, platinum black, among others.

Electrodes 162, 164 and 165 may be positioned at locations along pacemaker 14 other than the locations shown. Examples of various pacing electrode arrangements for providing cardiac pacing along the native conduction system of the heart and/or ventricular myocardium are generally disclosed in U.S. Publication No. 2019/0083779 (Yang, et al., granted as U.S. Pat. No. 11,426,578), and U.S. Pat. No. 11,007,369 (Sheldon, et al.), both of which are incorporated herein by reference in their entirety.

When tip electrode 164 and ring electrode 162 are used for sensing ventricular R-waves from an RA implant location, atrial P-waves may be oversensed as false R-waves. When ring electrodes 162 and 165 are used for sensing atrial P-waves, far field R-waves may be oversensed as false P-waves. These types of cross-chamber oversensing may occur when the electrodes used for sensing a cardiac electrical signal from one heart chamber are in close proximity to another heart chamber, e.g., as in the case of pacemaker 14 positioned in the RA for delivering ventricular pacing. As described herein, pacemaker 14 may be configured to sense both P-waves and R-waves in some examples for controlling ventricular pacing pulse delivery in various pacing modes, which may include both atrial synchronous ventricular pacing modes and asynchronous pacing modes. Whether pacemaker 14 is configured to sense ventricular R-waves, atrial P-waves or both, pacemaker 14 may be configured to confirm sensed cardiac event signals according to the techniques disclosed herein to avoid a CCOS event signal from interfering with the scheduling and delivery of cardiac pacing pulses according to an operating pacing mode.

Housing 150 is formed from a biocompatible material, such as a stainless steel or titanium alloy. In some examples, the housing 150 may include an insulating coating. Examples of insulating coatings include parylene, urethane, PEEK, or polyimide, among others. The entirety of the housing 150 may be insulated, but only electrodes 162, 164 and 165 uninsulated. Tip electrode 164 may serve as a cathode electrode and can be coupled to internal circuitry, e.g., a pacing pulse generator and cardiac electrical signal sensing circuitry, enclosed by housing 150 via an electrical feedthrough crossing housing 150. Electrodes 162 and 165 may be formed as a conductive portion of housing 150 defining respective ring electrodes that are electrically isolated from each other and from the other portions of the housing 150 as generally shown in FIG. 2.

Pacemaker 14 may include features for facilitating deployment to and fixation at an implant site. For example, pacemaker 14 may optionally include a delivery tool interface 158. Delivery tool interface 158 may be located at the proximal end 104 of pacemaker 14 and is configured to connect to a delivery device, such as a catheter, used to position pacemaker 14 at an implant location during an implantation procedure. The delivery tool interface may enable a clinician to advance, retract and steer pacemaker 14 to an implant site and rotate pacemaker 14 to advance the helical tip electrode 164 into the cardiac tissue. Helical tip electrode 164 in this example provides fixation of pacemaker 14 at the implant site. In other examples, however, pacemaker 14 may include a set of fixation tines or other fixation members to secure pacemaker 14 to cardiac tissue. Numerous types of active and/or passive fixation members may be employed for anchoring or stabilizing pacemaker 14 in an implant position.

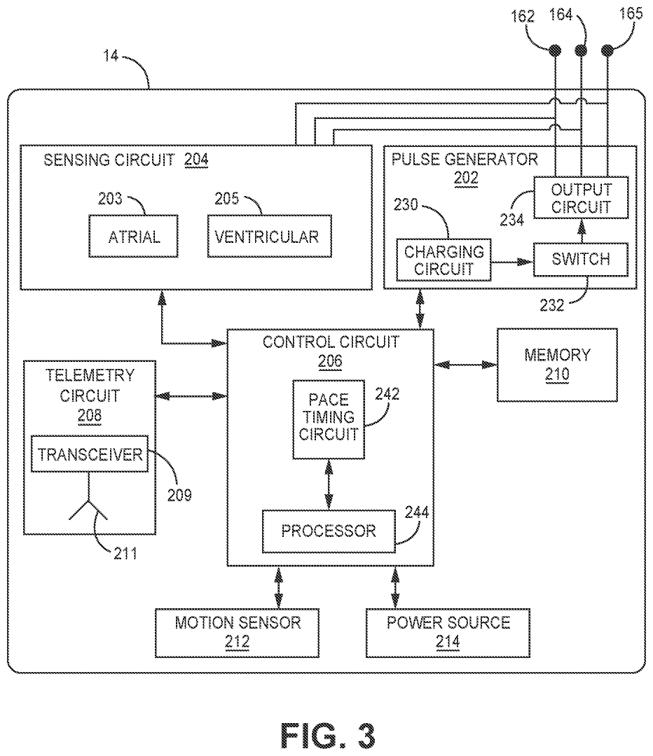

FIG. 3 is a conceptual diagram of an example configuration of pacemaker 14 according to some examples. Pacemaker 14 may include a pulse generator 202, a cardiac electrical signal sensing circuit 204, a control circuit 206, telemetry circuit 208, memory 210, motion sensor 212 and a power source 214. The various circuits represented in FIG. 3 may be combined on one or more integrated circuit boards which include an application specific integrated circuit (ASIC), an electronic circuit, a processor (shared, dedicated, or group) and memory that execute one or more software or firmware programs, a combinational logic circuit, state machine or other suitable components that provide the described functionality.

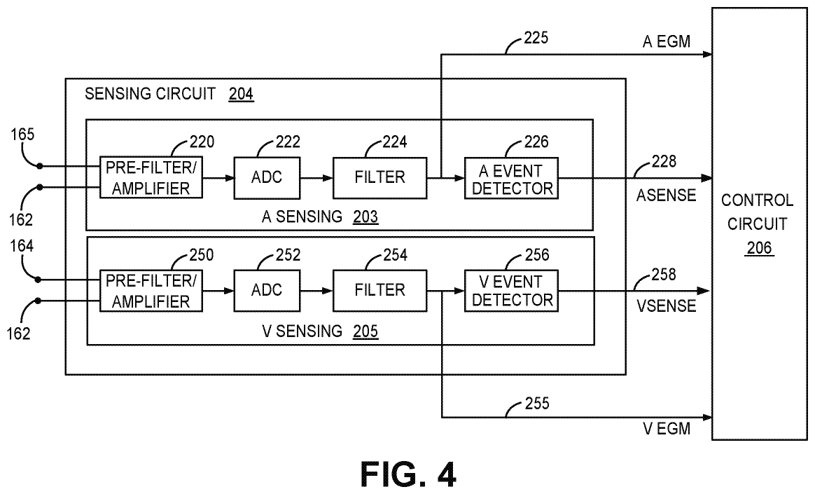

Cardiac electrical signal sensing circuit 204, referred to hereafter as “sensing circuit” 204, is configured to receive at least one cardiac electrical signal via electrodes coupled to pacemaker 14, e.g., via tip electrode 164 and ring electrode 162. When ring electrode 165 is present, another cardiac electrical signal may be received via electrodes 162 and 165 and/or electrodes 164 and 165. As such, sensing circuit 204 may have multiple sensing channels, e.g., an atrial sensing channel 203 and a ventricular sensing channel 205. While pacemaker 14 is shown having three electrodes in the examples shown herein, pacemaker 14 may be provided with two electrodes or more than three electrodes in other examples, which may be coupled to sensing circuit 204 (and/or pulse generator 202) in selected sensing (and/or pacing) electrode vectors. Sensing circuit 204 may include switching circuitry for coupling a sensing electrode pair to a respective sensing channel 203 or 205. As described below in conjunction with FIG. 4, sensing channels 203 and 205 may include filters, amplifiers, analog-to-digital converters (ADCs), rectifiers, sense amplifiers, comparators, and/or other circuitry for sensing cardiac event signals, e.g., P-waves and/or R-waves, and producing sensed cardiac event signals, e.g., atrial sensed event signals (Asense signals) and ventricular sensed event signals (Vsense signals), that are passed to control circuit 206. Sensing circuit 204 may be configured to pass a filtered and amplified multi-bit digital cardiac electrogram (EGM) signal to control circuit 206, e.g., from one or both of atrial and ventricular sensing channels 203 and 205. The EGM signal(s) may be processed and analyzed by control circuit 206 for determining a heart rhythm and/or stored in memory 210 as cardiac signal episodes that can be transmitted by telemetry circuit 208, e.g., to external device 20 (shown in FIG. 1).

Control circuit 206 may include a pace timing circuit 242 and processor 244. As described below in conjunction with FIG. 4, control circuit 206 may receive Vsense signals and Asense signals from sensing circuit 204 for use in controlling the timing of cardiac pacing pulses. Vsense signals may be passed from sensing circuit 204 to control circuit 206 in response to ventricular sensing channel 205 sensing a ventricular event signal to indicate the timing of a suspected R-wave. Asense signals may be passed from sensing circuit 204 to control circuit 206 in response to atrial sensing channel 203 sensing an atrial event signal to indicate the timing of a suspected P-wave.

Processor 244 may pass sensing control parameters to sensing circuit 204 for use in sensing cardiac event signals from the cardiac electrical signal(s). For example, one or more blanking periods, refractory periods, atrial sensitivity, ventricular sensitivity, and other control parameters used by sensing circuit 204 for applying a sensing threshold amplitude for sensing cardiac event signals may be passed to sensing circuit 204 from processor 244. Techniques for sensing cardiac event signals and confirming sensed cardiac event signals as being atrial or ventricular event signals are further described below.

Processor 244 may include one or more clocks for generating clock signals that are used by pace timing circuit 242 to time out various pacing intervals for providing ventricular pacing according to an operating pacing mode. Depending on the operating pacing mode of control circuit 206, pace timing circuit 242 may start a pacing interval to schedule a pacing pulse. Control circuit 206 may be configured to operate in a variety of programmable and/or automatically switchable pacing modes. During an atrial synchronous ventricular pacing mode, which may be denoted as a DDD or VDD pacing mode for example, ventricular pacing pulses may be delivered synchronously with atrial pacing pulses and received Asense signals. For example, in response to receiving an Asense signal, pace timing circuit 242 may start an AV pacing interval to control the timing of an atrial synchronous ventricular pacing pulse. When a ventricular pacing pulse is delivered by pulse generator 202 upon expiration of the AV pacing interval, pace timing circuit 242 may start a ventricular pacing interval to schedule a ventricular pacing pulse. During atrial synchronous ventricular pacing, if an Asense signal is not received prior to the expiration of a ventricular pacing interval, pulse generator 202 may deliver an asynchronous pacing pulse and restart the ventricular pacing interval. The scheduled ventricular pacing pulse can be inhibited if an Asense signal is received (or an atrial pacing pulse is delivered) before the ventricular pacing interval expires. The pending pacing pulse may be cancelled and an atrial synchronous triggered ventricular pacing pulse can be delivered at the AV pacing interval from the Asense signal (or delivered atrial pacing pulse). In some examples, a scheduled pacing pulse or restarting of a pacing interval may be delayed as a pending pacing pulse until a received Asense or Vsense signal is confirmed or determined to be a CCOS signal based on analysis of the cardiac signals according to the techniques disclosed herein.

In response to receiving a Vsense signal from sensing circuit 204, pace timing circuit 242 may inhibit a pending ventricular pacing pulse scheduled at the ventricular pacing interval (or scheduled at an AV pacing interval) and restart the ventricular pacing interval. The ventricular pacing interval may be a lower rate interval (LRI) corresponding to a programmed minimum or base ventricular pacing rate. In other instances, the ventricular pacing interval may be a temporary ventricular pacing interval set to a rate smoothing interval to avoid an abrupt change in ventricular rate. In other instances, the ventricular pacing interval may be a temporary rate response pacing interval set to provide rate response pacing during increased patient physical activity, which may be determined from a signal from motion sensor 212 as further described below.

Pulse generator 202 generates electrical pacing pulses that can be delivered to pace the ventricles of the patient's heart via cathode electrode 164 and return anode electrode 162. In examples including atrial pacing capabilities, pulse generator 202 may generate electrical pacing pulses for pacing the atria, e.g., using electrodes 165 and 162. In addition to providing control signals to pace timing circuit 242 and pulse generator 202 for controlling the timing of pacing pulses, processor 244 may retrieve programmable pacing control parameters from memory 210, such as pacing pulse amplitude and pacing pulse width, which are passed to pulse generator 202 for controlling pacing pulse delivery.

Pulse generator 202 may include charging circuit 230, switching circuit 232 and an output circuit 234. Charging circuit 230 is configured to receive current from power source 214 and may include a holding capacitor that may be charged to a pacing pulse amplitude, e.g., under the control of a voltage regulator included in charging circuit 230. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of a pacing escape interval and kept closed for a programmed pacing pulse width to enable discharging of the holding capacitor of charging circuit 230. The holding capacitor, previously charged to the pacing pulse voltage amplitude, can be discharged across electrodes 164 and 162 (or 165 and 162) through an output capacitor of output circuit 234 for the programmed pacing pulse duration. Examples of pacing circuitry generally disclosed in U.S. Pat. No. 5,507,782 (Kieval, et al.) and in U.S. Pat. No. 8,532,785 (Crutchfield, et al.), both of which patents are incorporated herein by reference in their entirety, may be implemented in pacemaker 14 for charging a pacing capacitor to a predetermined pacing pulse amplitude under the control of control circuit 206 and delivering a pacing pulse.

It is to be understood that when pacemaker 14 is configured for dual chamber pacing, pulse generator 202 may be configured for delivering both atrial and ventricular pacing pulses under the control of pace timing circuit 242. The atrial pacing pulses are generated by pulse generator 202 according to an atrial pacing pulse amplitude and pulse width. The ventricular pacing pulses are generated by pulse generator 202 according to a ventricular pacing pulse amplitude and pulse width. Pulse generator 202 may include an atrial pacing channel and a ventricular pacing channel that may be controlled separately to deliver atrial pacing pulses upon expiration of atrial pacing intervals and ventricular pacing pulses upon expiration of AV pacing intervals and/or ventricular pacing intervals. The separate atrial pacing channel and ventricular pacing channel may include some shared circuitry for generating and delivering pacing pulses. For example, atrial and ventricular pacing channels may include shared output circuitry that is selectively coupled to the appropriate pacing electrode pair via switching circuitry included in output circuit 234.

Pacemaker 14 may include a motion sensor 212, e.g., an accelerometer, for sensing patient motion. Motion sensor 212 may include a single-axis or multi-axis accelerometer for producing acceleration signals in one or more dimensions, which can be used for determining a relative level of patient physical activity. In some examples, pacemaker 14 may be capable of delivering rate response pacing based on a patient physical activity metric determined from an acceleration signal produced by motion sensor 212. Control circuit 206 may receive a rectified acceleration signal from motion sensor 212 and determine a patient physical activity metric from the acceleration signal, e.g., by summing acceleration signal sample point amplitudes over an activity metric time interval. The activity metric may be converted to a target heart rate to meet the patient's metabolic demand. The target heart rate may be converted to a sensor indicated rate (SIR) based on an SIR transfer function that includes a lower rate set point and an activities of daily living (ADL) range and a maximum upper rate, for example. During a rate response pacing mode, pulse generator 202 may be controlled by control circuit 206 to deliver atrial or ventricular pacing pulses at a rate response pacing rate determined based on the SIR. Examples of methods for providing rate response pacing based on an acceleration signal are generally disclosed in U.S. Pat. No. 9,724,518 (Sheldon, et al.), incorporated herein by reference in its entirety.

Memory 210 may include computer-readable instructions that, when executed by control circuit 206, cause control circuit 206 to perform various functions attributed throughout this disclosure to pacemaker 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal.

Memory 210 may store sensed event data based on Vsense signals and Asense signals received from sensing circuit 204 and may flag Vsense signals and/or Asense signals as false event signals when identified as CCOS event signals according to the techniques disclosed herein. In some examples, memory 210 includes a buffer that stores one or more episodes of EGM signals received from sensing circuit 204. Memory 210 may store one or more episodes of EGM signals when CCOS event signals are identified. A history of CCOS event signals and a representative EGM signal episode may be stored in memory 210 for transmission via telemetry circuit 208.

Telemetry circuit 208 includes a transceiver 209 and antenna 211 for transferring and receiving data via a radio frequency (RF) communication link. Telemetry circuit 208 may be capable of bi-directional communication with external device 20 (FIG. 1) as described above. Cardiac electrical signals and/or data derived therefrom such as sensed event data may be transmitted by telemetry circuit 208 to external device 20. Programmable control parameters and algorithms for sensing cardiac event signals and for confirming sensed event signals and identifying CCOS event signals as well as for controlling pacing therapies delivered by pulse generator 202 may be received by telemetry circuit 208 and stored in memory 210 for access by control circuit 206.

Power source 214 provides power to each of the other circuits and components of pacemaker 14 as required. Power source 214 may include one or more energy storage devices, such as one or more rechargeable or non-rechargeable batteries. The connections between power source 214 and other pacemaker circuits and components are not shown in FIG. 3 for the sake of clarity but are to be understood from the general block diagram of FIG. 3. Power source 214 may provide power as needed to pulse generator 202, sensing circuit 204, telemetry circuit 208, memory 210 and motion sensor 212.

The functions attributed to pacemaker 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, a process for confirming a cardiac event signal may be implemented in control circuit 206 executing instructions stored in memory 210 and relying on input from sensing circuit 204. Providing software, hardware, and/or firmware to accomplish the described functionality in the context of any modern pacemaker, given the disclosure herein, is within the abilities of one of skill in the art.

FIG. 4 is a conceptual diagram of sensing circuitry of pacemaker 14 according to some examples. Sensing circuit 204 may include atrial sensing channel 203 and ventricular sensing channel 205. Atrial sensing channel 203 is shown to receive a raw cardiac electrical signal from electrodes 165 and 162. Ventricular sensing channel 205 is shown to receive a raw cardiac electrical signal from electrodes 164 and 162. However, it is to be understood that other sensing electrode vectors may be provided as input to sensing channels 203 and/or 205 depending on what electrodes are coupled to or included on pacemaker housing 150 and which sensing electrode vectors provide the greatest sensitivity for sensing P-waves and R-waves and the greatest discrimination between true P-waves and true R-waves. For instance, ventricular sensing channel 205 may receive a raw cardiac electrical signal from electrodes 164 and 165 in some examples.

The raw cardiac electrical signals are received as input to a pre-filter and amplifier circuit 220 or 250 of atrial sensing channel 203 or ventricular sensing channel 205, respectively. Pre-filter and amplifier circuits 220 and 250 may each include a high pass filter to remove DC offset, e.g., a 2.5 to 5 Hz high pass filter, or a wideband filter having a bandpass of 2.5 Hz to 100 Hz or narrower to remove DC offset and high frequency noise. Pre-filter and amplifier circuits 220 and 250 may further include an amplifier to amplify the raw cardiac electrical signal passed to a respective ADC 222 or 252.

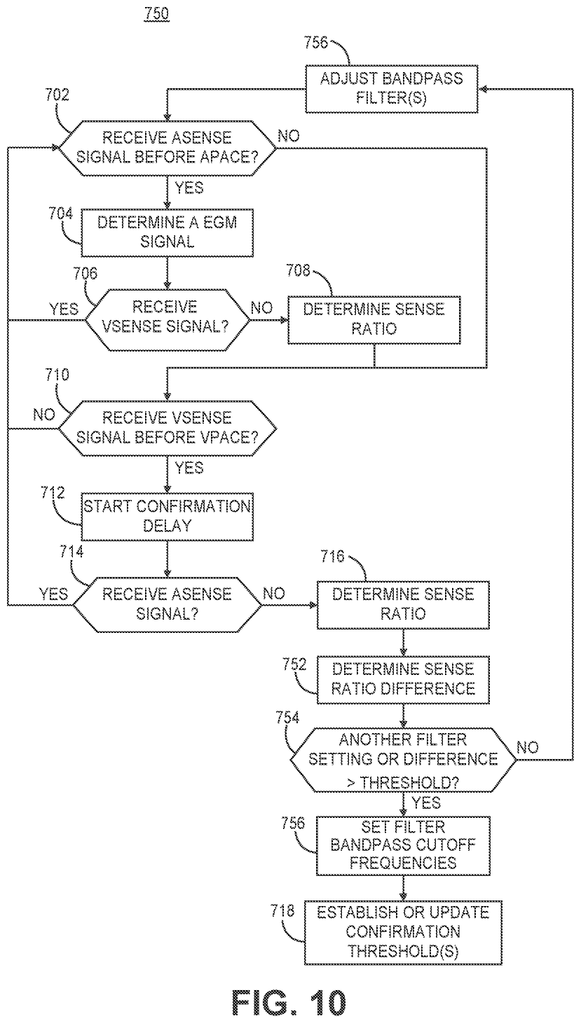

ADC 222 may pass a rectified digital signal to filter 224 of atrial sensing channel 203. Filter 224 may be a bandpass filter having bandpass cutoff frequencies for passing P-wave signals and attenuating other cardiac event signals, e.g., R-waves and T-waves. In some examples, the high pass and/or low pass cutoff frequencies of filter 224 are user programmable and/or may be adjusted by control circuit 206 to reduce the incidence of CCOS of far-field R-waves by atrial sensing channel 203 and/or improve the discrimination between true sensed event signals and CCOS event signals. An atrial EGM signal 225 filtered according to different cutoff frequency settings applied by filter 224 may be passed to control circuit 206 for use in selecting the optimal bandpass cutoff frequencies of filter 224 for promoting reliable sensing of atrial P-waves. In other examples, the output of ADC 222 may be passed to control circuit 206 for analysis for selecting the optimal low pass and high pass cutoff frequencies of filter 224. Methods for selecting the high pass and low pass filter frequencies are described below in conjunction with FIG. 10. The low pass cutoff frequency of filter 224 may be between 25 Hz and 100 Hz or between 50 and 80 Hz and the high pass cutoff frequency may be between 5 Hz and 25 Hz or between 15 and 20 Hz, as examples. In an example, the filter 224 is provided with a low pass frequency of 70 Hz and a high pass frequency of 17 Hz.

Atrial event detector 226 may include a sense amplifier, comparator or other event detection circuitry that compares the incoming rectified, filtered and amplified atrial EGM signal to a P-wave sensing threshold. For example, when the incoming signal crosses a P-wave sensing threshold, the atrial event detector 226 produces an atrial sensed event signal (Asense) 228 that can be passed to control circuit 206. The P-wave sensing threshold may be an auto-adjusting threshold that is automatically decreased by sensing circuit 204 from a starting value to a minimum value or until a P-wave sensing threshold crossing occurs. The P-wave sensing threshold amplitude is initially set to a starting value applied to the atrial EGM signal upon expiration of a post-atrial blanking period and can be adjusted to a minimum sensing threshold or “sensing floor” that may be equal to a programmed atrial sensitivity.

Atrial event detector 226 may include a peak track and hold circuit or other circuitry for detecting the maximum peak amplitude of the atrial EGM signal during a portion of the post-atrial blanking period. The P-wave sensing threshold starting value may be set based on the maximum peak amplitude, e.g., to a percentage of the maximum peak amplitude following a P-wave sensing threshold crossing. In some examples, the P-wave sensing threshold may be set to 50% to 80% of the maximum peak amplitude. The P-wave sensing threshold may be decreased according to one or more decay rates and corresponding decay time intervals until the atrial EGM signal crosses the P-wave sensing threshold or the atrial sensitivity is reached. The atrial sensitivity defines the minimum atrial EGM signal amplitude that can be sensed as a P-wave In other examples, the P-wave sensing threshold may be a fixed value that does not decay over time and may be applied as a percentage of the maximum peak amplitude of the atrial EGM signal during a post-atrial blanking period or based on the programmed atrial sensitivity. If an atrial pacing interval, which may be set to a programmed LRI expires before an Asense signal 228 is received by control circuit 206, pulse generator 202 (shown in FIG. 3) may deliver an atrial pacing pulse in some examples.

ADC 252 of ventricular sensing channel 205 may pass a rectified signal to filter 254. Filter 254 may be a bandpass filter having bandpass cutoff frequencies for passing R-wave signals and attenuating other cardiac event signals, e.g., P-waves and T-waves. In some examples, the high pass and/or low pass cutoff frequencies of filter 254 are user programmable and/or may be adjusted by control circuit 206 to reduce the incidence of CCOS of P-waves by ventricular sensing channel 205 and/or improve the discrimination between true sensed event signals and CCOS event signals, as further described below. A ventricular EGM signal 255 filtered according to different cutoff frequency settings applied by filter 254 may be passed to control circuit 206 for use in selecting the optimal bandpass cutoff frequencies of filter 254 for promoting reliable sensing of R-waves. In other examples, the output of ADC 252 may be passed to control circuit 206 for analysis for selecting the optimal low pass and high pass cutoff frequencies of filter 254. Methods for selecting the high pass and low pass filter frequencies are described below in conjunction with FIG. 10. The low pass cutoff frequency of filter 254 may be between 25 Hz and 100 Hz or between 40 and 80 Hz and the high pass cutoff frequency may be between 5 Hz and 25 Hz or between 15 and 20 Hz, as examples. In an example, the filter 224 is provided with a low pass frequency of 70 Hz and a high pass frequency of 17 Hz. The bandpass cutoff frequencies of filter 254 may be different than the bandpass cutoff frequencies of filter 224.

Ventricular event detector 256 may include a sense amplifier, comparator or other event detection circuitry that compares the incoming rectified, filtered and amplified ventricular EGM signal to an R-wave sensing threshold. For example, when the incoming signal crosses an R-wave sensing threshold, the ventricular event detector 256 may produce a ventricular sensed event signal (Vsense) 256 that can be passed to control circuit 206. The R-wave sensing threshold may be an auto-adjusting threshold that is automatically decreased by sensing circuit 204. The R-wave sensing threshold amplitude is initially set to a starting value applied to the ventricular EGM signal upon expiration of a post-ventricular blanking period and can be adjusted to a minimum sensing threshold or “sensing floor” that may be equal to a programmed ventricular sensitivity.

Ventricular event detector 256 may include a peak track and hold circuit or other circuitry for detecting the maximum peak amplitude of the ventricular EGM signal following an R-wave sensing threshold crossing during a peak tracking portion of the post-ventricular blanking period. The R-wave sensing threshold starting value may be set based on the maximum peak amplitude, e.g., to a percentage of the maximum peak amplitude. In some examples, the R-wave sensing threshold may be set to 50 to 80% of the maximum peak amplitude. The R-wave sensing threshold may be decreased according to one or more decay rates and corresponding decay time intervals until the ventricular EGM signal crosses the R-wave sensing threshold or the ventricular sensitivity is reached. The ventricular sensitivity defines the minimum ventricular EGM signal amplitude that can be sensed as an R-wave. In other examples, the R-wave sensing threshold may be a fixed value that does not decay, e.g., a fixed percentage of the maximum peak amplitude or a fixed value based on a programmed ventricular sensitivity. If a ventricular pacing interval or an AV pacing interval expires before a Vsense signal 258 is received by control circuit 206, pulse generator 202 (shown in FIG. 3) may deliver a ventricular pacing pulse.

Control circuit 206 may provide sensing control signals to sensing circuit 204. Sensing control parameters may include R-wave sensing threshold adjustment parameters, e.g., the percentage of the maximum peak amplitude used for setting the starting R-wave sensing threshold and the ventricular sensitivity, and P-wave sensing threshold adjustment parameters, e.g., the percentage of the maximum peak amplitude used for setting the starting P-wave sensing threshold and the atrial sensitivity. Sensing control parameters may include various blanking and refractory intervals applied to the atrial EGM signal, e.g., a post-sense atrial blanking period, a post-pace atrial blanking period, an atrial refractory period and a post-ventricular atrial blanking period. Sensing control parameters may include various blanking and refractory intervals applied to the ventricular EGM signal, e.g., a post-sense ventricular blanking period, a post-pace ventricular blanking period and a post-atrial ventricular blanking period.

As described above, Asense signals 228 and Vsense signals 258 passed from sensing circuit 204 to control circuit 206 may be used for controlling the timing of atrial and/or ventricular pacing pulses by pace timing circuit 242. Pace timing circuit 242 (shown in FIG. 3) may start a pacing escape interval timer upon receiving an Asense or Vsense signal. The value reached by an escape interval timer between two consecutive Asense signals or between an Asense signal and a preceding atrial pacing pulse can be determined as a PP interval (PPI) for use in determining an atrial rate. The value reached by an escape interval timer between two consecutive Vsense signals or between a Vsense signal and a preceding ventricular pacing pulse can be determined as an RR interval (RRI) for use in determining a ventricular rate. The atrial rate and/or ventricular rate may be determined by control circuit 206 for storing cardiac data in memory 210, controlling pacing mode switching, or other pacemaker functions in some examples.

When sensing circuit 204 is configured to receive a raw atrial electrical signal and a raw ventricular electrical signal as shown in FIG. 4, components included in an atrial sensing channel 203 and in a ventricular sensing channel 205 may be separate or shared between both sensing channels 203 and 205 in various examples. For example, pre-filter/amplifiers 220 and 250 and/or ADCs 222 and 252 may be shared by both atrial sensing channel 203 and ventricular sensing channel 205 with separate outputs being passed to atrial event detector 226, e.g., via filter 224, and to ventricular event detector 256, e.g., via filter 254. Different filtering and amplification may be applied to the output of an ADC before passing separate signals to the respective atrial event detector 226 and ventricular event detector 256. For example filter 224 can be tuned to enhance P-wave signal amplitude and attenuate other cardiac signals. Filter 254 can be tuned to enhance R-wave signal amplitude and attenuate other cardiac signals.

Furthermore, it is to be understood that other sensing electrode vectors than the vectors shown in FIG. 4 may be used in discriminating between true cardiac sensed event signals and oversensed signals according to the techniques disclosed herein. In the example of pacemaker 14 including tip electrode 164 and two ring electrodes 162 and 165, any combination of the sensing electrode vectors, e.g., between electrodes 164 and 162, electrodes 164 and 165, and/or electrodes 162 and 165 may be received as input to sensing circuit 204 for use in discriminating between true cardiac sensed event signals and oversensed signals according to the techniques disclosed herein. As described below, control circuit 206 may be configured to determine a quantitative relationship between a feature of a ventricular EGM signal and an analogous feature of an atrial EGM signal. In illustrative examples presented herein the quantitative relationship is determined as a ratio of a feature of a ventricular EGM signal and an analogous feature of an atrial EGM signal and is referred to as a “sense ratio.” The quantitative relationship may be used in discriminating between CCOS events and true events sensed by a sensing channel 203 or 205. The quantitative relationship may be determined by dividing, multiplying, adding and/or subtracting the feature of the ventricular EGM signal and the analogous feature of the atrial EGM signal. In various examples, the quantitative relationship could be a “sense ratio,” a “sense product,” a “sense difference” or a “sense sum” that is respectively determined by dividing, multiplying, subtracting or adding the ventricular EGM signal feature and atrial EGM signal feature. The quantitative relationship may be determined using weighting factors in some examples. The quantitative relationship may be determined as any combination of dividing, multiplying, subtracting and/or adding the values of ventricular EGM signal feature and the atrial EGM signal feature. The EGM signal that is passed to atrial event detector 226 and an EGM signal used to determine a signal feature corresponding to a P-wave sensed by atrial event detector 226 and used for calculating a quantitative relationship, e.g., the sense ratio or any of the examples listed above, may be the same or two different sensing electrode vectors. The EGM signal that is passed to ventricular event detector 256 and the EGM signal used to determine a signal feature corresponding to an R-wave sensed by ventricular event detector 256 for calculating a quantitative relationship, e.g., sense ratio, may be sensed using the same or two different sensing electrode vectors. Accordingly, sensing circuitry included in a medical device configured to operate according to the presently disclosed techniques include one or more sensing circuits, which may be configured as multiple sensing channels, having separate or shared components for filtering, amplifying, digitizing and/or rectifying two or more raw cardiac electrical signals for sensing P-wave sensing threshold crossings, sensing R-wave sensing threshold crossings, determining atrial EGM signal features associated with Asense signals 228 and determining ventricular EGM signal features associated with a Vsense signals 258. The sensing circuitry and the control circuit 206 may individually or cooperatively determine one or more signal features corresponding to the time of the P-wave sensing threshold crossings and determine one or more signal features corresponding to the time of the R-wave sensing threshold crossings.

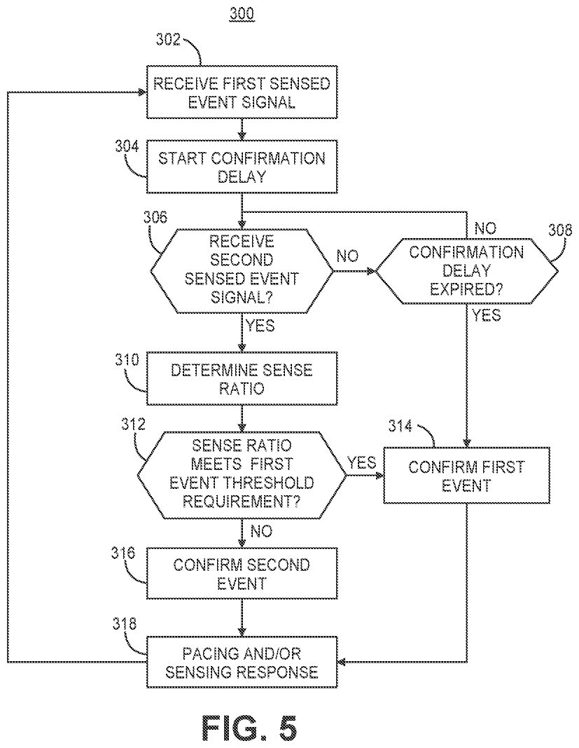

FIG. 5 is a flow chart 300 of a method that can be performed by pacemaker 14 for confirming a sensed cardiac event signal and identifying a CCOS event signal. At block 302, control circuit 206 receives a first signal corresponding to a cardiac event signal. With continued reference to FIG. 4, the first signal may be a sensed event signal, either an Asense signal or a Vsense signal, from sensing circuit 204 indicating the time of a P-wave sensing threshold crossing or an R-wave sensing threshold crossing, respectively. In other examples, processing circuitry of control circuit 206, e.g., processor 244, may be configured to receive the atrial EGM signal 225 and the ventricular EGM signal 255 from sensing circuit 204 (see FIG. 4). Control circuit 206 may receive a first sensed event signal by receiving an EGM signal and identifying the cardiac event, e.g., P-wave or R-wave, from the respective atrial EGM signal or ventricular EGM signal. The cardiac event may be identified from the respective EGM signal based on the amplitude, slope, morphology or other characteristics of the cardiac waveform.

In response to receiving the first sensed event signal at block 302, control circuit 206 may start a confirmation delay at block 304. The confirmation delay is a time interval that can be started by control circuit 206, e.g., by starting a timer in processor 244. The confirmation delay may be set to 10 milliseconds (ms), 20 ms, 30 ms, 50 ms, 70 ms, 100 ms or other time interval. The confirmation delay may be programmable by a user. The confirmation delay may be rate adaptive such that it may be adjusted to a shorter delay as heart rate increases and longer delay as heart rate decreases. The heart rate may be determined by control circuit 206 based on a mean, median, or other representative value of the most recent specified number of PPIs or RRIs, e.g., the most recent 8 to 20 PPIs determined between confirmed Asense signals and/or atrial pacing pulses or the most recent 8 to 20 RRIs determined between confirmed Vsense signals and/or ventricular pacing pulses.

The first sensed event signal may be a “true” cardiac event sensed from the intended heart chamber (atrial or ventricular) or a CCOS event occurring in the other heart chamber (ventricular or atrial) that is sensed by one sensing channel of sensing circuit 204. For example, if the first sensed event signal is an Asense signal received from atrial sensing channel 203, the first sensed event signal could be a CCOS far-field R-wave that is subsequently sensed as a true R-wave by ventricular sensing channel 205 within 10 to 100 ms after the far-field R-wave crosses the P-wave sensing threshold of atrial sensing channel 203. In other instances, if the first sensed event signal is an Asense signal received from atrial sensing channel 203, the first sensed event signal could be a true P-wave that is subsequently oversensed by ventricular sensing channel 205 as a false R-wave, resulting in a false Vsense signal within 10 to 100 ms after the Asense signal.

In other instances the first sensed event signal received at block 302 may be a Vsense signal. The Vsense signal may be a true Vsense signal but can be followed by a false Asense signal within the confirmation delay due to CCOS of a far-field R-wave by the atrial sensing channel 203. The Vsense signal may be a false Vsense signal due to oversensing of a large P-wave, that may be subsequently sensed by the atrial sensing channel 203.

The confirmation delay started at block 304 may be a fixed delay regardless of whether an Asense event signal is received first at block 302 or a Vsense event signal is received first at block 302. In other examples, the confirmation delay may be set differently, e.g., shorter or longer, when the first sensed event signal received at block 302 is an Asense event signal than when the first sensed event signal received at block 302 is a Vsense event signal. Depending on the filtering, amplification, P-wave and R-wave sensing thresholds and other factors, the maximum time interval within which a CCOS event is expected to occur on one sensing channel relative to the true sensed event on the other sensing channel may be different.

Control circuit 206 may determine at block 306 if a second sensed event signal is received before the confirmation delay expires (as determined at block 308). If a second sensed event signal is not received before the confirmation delay expires (“yes” branch of block 308), control circuit 206 may confirm the first sensed event signal as a true sensed event at block 314. If the first sensed event signal corresponds to a sensed P-wave, the sensed P-wave is confirmed as a true event at block 314. If the first sensed event signal corresponds to a sensed R-wave, the sensed R-wave can be confirmed at block 314.

Control circuit 206 may perform a pacing and/or sensing response to the confirmed sensed event signal at block 318. If the confirmed event is a P-wave, control circuit 206 may start an AV pacing interval, an atrial LRI, a post-sense atrial blanking period, an atrial refractory period, and a post-atrial ventricular blanking period as examples. A pending atrial pacing pulse, e.g., scheduled at an expiration of an atrial LRI that is running at the time of receiving the first sensed event signal at block 302, may be inhibited, and the atrial LRI may be restarted. A ventricular pacing escape interval that is running at the expiration of the confirmation delay may continue to run when the confirmed event is a P-wave. If a ventricular pacing escape interval expires during the confirmation delay, the pending ventricular pacing pulse can be delivered upon expiration of the confirmation delay when the confirmed event is a P-wave. In other examples, control circuit 206 may additionally or alternatively control telemetry circuit 208 to transmit an atrial event communication signal when the confirmed event is a P-wave. The atrial event signal may be received by another medical device, e.g., an intracardiac leadless ventricular pacemaker, that is triggered to deliver a ventricular pacing pulse in response to receiving the transmitted atrial event signal.

If the confirmed event is an R-wave at block 314, control circuit 206 may start various pace timing and/or sensing control intervals such as a ventricular LRI (which may be set to a temporary rate response or rate smoothing interval), a post-sense ventricular blanking period, a ventricular refractory period, a post-ventricular atrial blanking period and a post-ventricular atrial refractory period, as examples. If the confirmed event is an R-wave, control circuit 206 may inhibit a pending ventricular pacing pulse scheduled to be delivered upon expiration of an AV pacing interval and/or a ventricular pacing interval that is running at the time that the first sensed event signal is received at block 302. The ventricular pacing interval may be restarted in response to confirming the first event as a true R-wave. An atrial pacing escape interval that is running at the expiration of the confirmation delay may continue to run when the confirmed event is an R-wave. If an atrial pacing escape interval expires during the confirmation delay, the pending atrial pacing pulse can be delivered upon expiration of the confirmation delay when the confirmed event is an R-wave. In other examples, control circuit 206 may additionally or alternatively control telemetry circuit 208 to transmit a ventricular event communication signal when the confirmed event is an R-wave. The transmitted ventricular event communication signal may be received by another medical device, e.g., an intracardiac leadless atrial pacemaker, an ICD or other medical device, that may use the received ventricular event communication signal in determining a heart rhythm, e.g., for arrhythmia detection, and/or for controlling delivered electrical stimulation therapies.

When a second sensed event signal is received by control circuit 206 at block 306 prior to the confirmation delay expiring at block 308 (“yes” branch of block 306), control circuit 206 may determine a quantitative relationship between a feature of the atrial EGM signal 225 received from the atrial sensing channel and a feature of the ventricular EGM signal 255 received from the ventricular sensing channel 205 (see FIG. 4). In the example shown, control circuit 206 determines a sense ratio at block 310. The sense ratio may be determined as a ratio of a feature determined from the atrial EGM signal 225 and a feature determined from the ventricular EGM signal 255. In some examples, the ratio is a ratio of the maximum peak amplitudes of the respective signals. The maximum peak amplitude may be determined from the atrial EGM signal and the ventricular EGM signal following the sensed event signal received at block 302, e.g., within the confirmation delay. In other examples, the maximum peak amplitudes may be determined from the respective EGM during a post-sense blanking period (or a peak tracking portion thereof) that is applied by the respective sensing channel. As described above, atrial event detector 226 and ventricular event detector 256 may each include a peak track and hold circuit or other components that determine the maximum peak amplitude of the respective atrial EGM and ventricular EGM signals following a respective P-wave sensing threshold crossing and R-wave sensing threshold crossing. Sensing circuit 204 may be configured to output the atrial EGM maximum peak amplitude following a P-wave sensing threshold crossing to control circuit 206. Sensing circuit 204 may be configured to output the ventricular EGM maximum peak amplitude following an R-wave sensing threshold crossing to control circuit 206. In other examples, control circuit 206 may be configured to determine the maximum peak amplitude of a respective atrial EGM signal 225 or ventricular EGM signal 255 received from sensing circuit 204 following an Asense or Vsense signal or in response to identifying a P-wave or R-wave from the respective EGM signals.

In other examples, the sense ratio may be a ratio of a different feature of the EGM signals. For example, the sense ratio may be a ratio of maximum slopes, signal widths, signal areas, or other features of the atrial EGM and ventricular EGM signals that are determined from each respective EGM signal following the first sensed event signal and/or the second sensed event signal. In still other examples, control circuit 206 may determine more than one sense ratio of more than one feature of the atrial and ventricular EGM signals. For example, a ratio of maximum peak amplitudes and a ratio of maximum peak slopes may be determined as sense ratios at block 310.

For the sake of illustration, the quantitative relationship is described in examples presented in conjunction with the accompanying drawings as being a sense ratio of the maximum peak amplitudes. However, it is to be understood that, in other examples, instead of determining the quantitative relationship as a ratio between a feature of the atrial EGM signal and a feature of the ventricular EGM signal, the quantitative relationship may be determined as a difference between an atrial EGM signal feature and an analogous ventricular EGM signal feature. The quantitative relationship may be determined as a maximum peak difference, a maximum slope difference, a signal width difference, a signal area difference or the difference between another feature of the atrial and ventricular EGM signals. In still other examples, the quantitative relationship may be determined as a sum, e.g., a maximum peak sum, a maximum slope sum, a signal width sum, a signal area sum or the sum of another feature of each of the atrial and ventricular EGM signals. In other examples, the quantitative relationship may be determined as a product, e.g., a maximum peak product, a maximum slope product, a signal width product, a signal area product or the product of another feature of each of the atrial and ventricular EGM signals. In some examples, the quantitative relationship may be a normalized ratio, a normalized product, a normalized difference or a normalized sum between an atrial EGM signal feature and a ventricular EGM signal feature by normalizing the quantitative relationship by a selected reference value. The quantitative relationship can be determined by control circuit 206 for performing a comparative analysis of two signals sensed by sensing circuit 204 within the confirmation delay from each other for discriminating between a true sensed event (that is intended to be sensed) and a CCOS event (that is not intended to be sensed by the given sensing channel). The quantitative relationship can be determined by control circuit 206 for performing a comparative analysis between the quantitative relationship and a discriminating threshold applied to the quantitative relationship that distinguishes between the relative magnitude of the quantitative relationship when the first event is a true event (intended to be sensed) and when the first event is a CCOS event (that is not intended to be sensed by the given sensing channel).

At block 312, control circuit 206 may compare the sense ratio to a threshold defined for confirming the first sensed event signal. In an example, the sense ratio is determined as the ratio of the maximum peak amplitudes. The sense ratio may be determined as the ratio of the maximum peak amplitude of the ventricular EGM signal determined following a Vsense signal (which may be the first or second sensed event signal received by control circuit 206) to the maximum peak amplitude of the atrial EGM signal determined following an Asense signal (which may be the second or first sensed event signal received by control circuit 206).

The sense ratio determined as the maximum amplitude of the ventricular EGM signal to the maximum amplitude of the atrial EGM signal can be referred to as the V/A sense ratio. For the sake of illustration, the examples described below refer to the V/A sense ratio as the sense ratio used for confirming a sensed event signal. However, it is to be understood that the sense ratio could be additionally or alternatively determined as the A/V ratio of the atrial EGM signal maximum peak amplitude to the ventricular EGM signal maximum peak amplitude. Furthermore the V/A sense ratio and/or the A/V sense ratio may be determined for other EGM signal features, such as slope, signal width, signal area, etc. as described above. It is further contemplated that the sense ratio may be determined as a V/A sense ratio or an A/V sense ratio depending on which sensed event signal is received first, Asense or Vsense, and which is received second, Vsense or Asense, within the confirmation delay. When both a V/A and an A/V sense ratio are determined, the V/A sense ratio and the A/V sense ratio may be determined using the same feature (e.g., maximum peak amplitudes) of the respective atrial EGM and ventricular EGM signals. In other examples, a V/A sense ratio may be determined using one feature, e.g., maximum amplitude, and an A/V sense ratio may be determined using a different feature, e.g., maximum slope, of the respective atrial and ventricular EGM signals. Accordingly, while the examples presented herein refer to a V/A sense ratio determined as a maximum amplitude ratio that may be compared to a P-wave confirmation threshold and/or an R-wave confirmation threshold, it is to be understood that one or more V/A sense ratios and/or one or more A/V ratios may be determined as ratios of amplitude and/or other EGM signal features for comparing to respective P-wave confirmation threshold(s) and/or R-wave confirmation threshold(s).

If the first sensed event signal is an Asense signal, control circuit 206 may compare the V/A sense ratio to a P-wave confirmation threshold at block 312. The V/A sense ratio can be determined as the maximum peak amplitude of the ventricular EGM following a P-wave sensing threshold crossing (or the subsequent Vsense signal that occurs within the confirmation delay) to the maximum peak amplitude of the atrial EGM following the same P-wave sensing threshold crossing. This V/A sense ratio is expected to be relatively low if the Asense signal is true and the Vsense signal received within the confirmation delay is a CCOS P-wave. If the Asense signal is false, the V/A sense ratio is expected to be a relatively high value due to a large R-wave peak amplitude in the ventricular EGM signal and relatively small amplitude of far-field R-wave in the filtered atrial EGM signal that is falsely oversensed as a P-wave. When the V/A sense ratio is less than (or equal to) a P-wave confirmation threshold at block 312, control circuit 206 may confirm the Asense signal received at block 302 as a true P-wave at block 314. In other examples, the maximum ventricular EGM signal amplitude may be required to be less than an R-wave threshold amplitude, and the maximum atrial EGM signal amplitude may be required to be greater than a P-wave threshold amplitude in order for control circuit 206 to confirm the Asense signal as a true P-wave at block 314 when the V/A sense ratio is less than the P-wave confirmation threshold.

When the V/A sense ratio is greater than the P-wave confirmation threshold at block 312, the Vsense signal received at block 306 may correspond to a true R-wave with the preceding Asense signal (received first at block 302) corresponding to a CCOS far-field R-wave. Control circuit 206 may confirm the second event signal received as a Vsense signal as a true R-wave at block 316. In other examples, the maximum ventricular EGM signal amplitude may be required to be greater than an R-wave threshold amplitude, and the maximum atrial EGM signal amplitude may be required to be less than a P-wave threshold amplitude in order for control circuit 206 to confirm the Vsense signal as a true R-wave at block 316.

If the first sensed event signal received at block 302 is a Vsense signal, control circuit 206 may compare the V/A sense ratio to an R-wave confirmation threshold at block 312. The V/A sense ratio determined as the maximum peak amplitude of the ventricular EGM following an R-wave sensing threshold crossing to the maximum peak amplitude of the atrial EGM following the same R-wave sensing threshold crossing (or the subsequent P-wave sensing threshold crossing that occurs within the confirmation delay) is expected to be relatively high if the Vsense signal is true and the Asense signal received within the confirmation delay is a CCOS far-field R-wave. If the V/A sense ratio is greater than an R-wave confirmation threshold at block 312, control circuit 206 may confirm the Vsense signal received at block 302 as a true R-wave at block 314.

When the V/A sense ratio is less than (or equal to) the R-wave confirmation threshold at block 312, the Vsense signal may correspond to a CCOS P-wave with the subsequent Asense signal occurring within the confirmation delay time corresponding to a true P-wave. Control circuit 206 may confirm the second event signal (received as an Asense signal) as a true P-wave at block 316 when the V/A sense ratio is less than (or equal to) the R-wave confirmation threshold and a Vsense signal was received prior to the Asense signal. The P-wave confirmation threshold and the R-wave confirmation threshold applied to the V/A sense ratio may be the same or different from each other.

When a true P-wave occurs, the V/A sense ratio may generally be less than 1.0 or less than 1.25 as examples. When a true R-wave occurs, the V/A sense ratio may generally be greater than 1.5, greater 2.0, in the range of 2.0 to 5.0, or may be as high as 10 or 15 as examples. Accordingly, the P-wave confirmation threshold may be equal to or between 1.0 and 1.5 and may be 1.25 in an example. The R-wave confirmation threshold may be set equal to the P-wave confirmation threshold. In other examples, the R-wave confirmation threshold may be higher, e.g., 1.5 to 2.0 or higher.

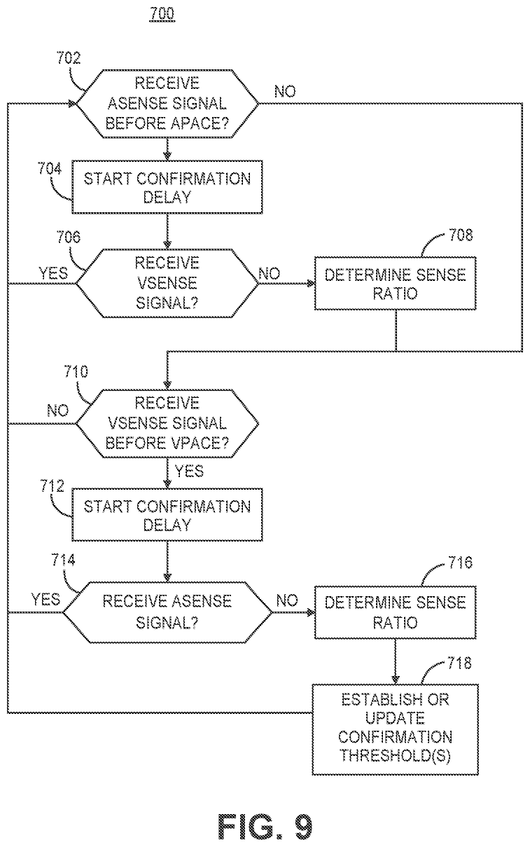

As described below in conjunction with FIG. 9, the threshold ratios for confirming a P-wave and an R-wave may be established by control circuit 206 based on an analysis of the atrial EGM signal and the ventricular EGM signal when a confident Asense signal is received and when a confident Vsense signal is received. After confirming either the first event or the second event as the true sensed event, control circuit 206 may perform a pacing and/or sensing response at block 318 as described above.

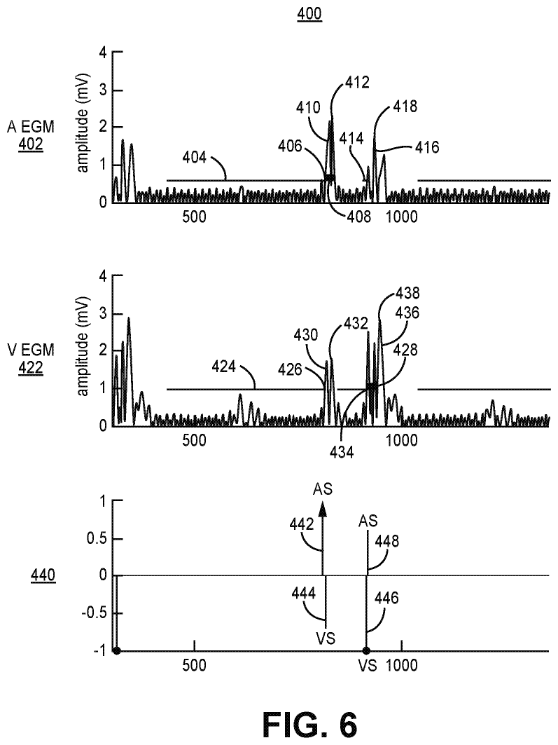

FIG. 6 is a timing diagram 400 of an atrial (A) EGM signal 402, a ventricular (V) EGM signal 422 and a sensed event marker channel 440 that depicts cardiac sensed event signals that may be received by control circuit 206 and confirmed as true events or identified as CCOS events by control circuit 206 according to some examples. Atrial sensing channel 203 may apply a P-wave sensing threshold 404 to the atrial EGM signal 402 for sensing atrial events. Atrial sensing channel 203 may produce an Asense signal 442 in response to a P-wave sensing threshold crossing 406. While the P-wave sensing threshold is shown as a fixed amplitude threshold in FIG. 6, which may correspond to the atrial sensitivity, it is to be understood that P-wave sensing threshold 404 may be an auto-adjusted threshold that may be adjusted from a starting amplitude that can be decreased to a minimum sensing threshold or until a P-wave sensing threshold crossing occurs.

In response to receiving the Asense event signal 442, control circuit 206 may start a confirmation delay 408. Ventricular sensing channel 205 may apply an R-wave sensing threshold 424 to the ventricular EGM signal 422. Ventricular sensing channel 205 may produce a Vsense signal 444 in response to the R-wave sensing threshold crossing 426. The R-wave sensing threshold 424 is shown as a fixed amplitude threshold, e.g., equal to the programmed ventricular sensitivity. However, it is to be understood that the R-wave sensing threshold 424 may be an auto-adjusting threshold that is decreased from a starting amplitude down to a minimum sensing threshold or until an R-wave sensing threshold crossing occurs.

Because the Vsense signal 444 occurs before expiration of the confirmation delay 408, control circuit 206 may determine the V/A sense ratio as the ratio of the ventricular EGM maximum peak amplitude 432 to the atrial EGM maximum peak amplitude 412. The ventricular EGM maximum peak amplitude 432 may be determined during the confirmation delay 408 or within a post-sense time interval, e.g., within 20 to 100 ms of the R-wave sensing threshold crossing 426. The atrial EGM maximum peak amplitude 412 may be determined as the maximum peak amplitude during the confirmation delay 408 or within a post-sense time interval of the P-wave sensing threshold crossing 406. The quantitative relationship between an atrial EGM signal feature and a ventricular EGM signal feature may alternatively be determined as a difference, sum or product or any combination thereof indicated above.

The V/A sense ratio (or other quantitative relationship) may be compared to a P-wave confirmation threshold by control circuit 206 because the Asense event signal 442 was received first and the Vsense event signal 444 was received second. The V/A sense ratio may be less than the P-wave confirmation threshold when the atrial EGM maximum peak amplitude 412 of the true P-wave 410 is relatively high and the ventricular EGM maximum peak amplitude 432 of the CCOS P-wave 430 is relatively low. In response to the V/A sense ratio being less than the P-wave confirmation threshold, control circuit 206 may confirm the Asense event signal 442 as a true P-wave. Control circuit 206 may identify the Vsense event signal 444 as a CCOS event signal. In other examples, the V/A sense ratio (or other quantitative relationship) may additionally or alternatively be compared to an R-wave confirmation threshold. If the V/A sense ratio is less than the R-wave confirmation threshold, control circuit 206 may confirm the Asense event signal 442 as being a true P-wave and identify the Vsense event signal 444 as a CCOS signal. As described above, control circuit 206 may provide a pacing and/or sensing response based on the confirmed P-wave.

Ventricular sensing channel 205 may produce another Vsense event signal 446 in response to R-wave sensing threshold crossing 434. Control circuit 206 may start the confirmation delay 428 in response to receiving Vsense event signal 446. An Asense event signal 448 is received by control circuit 206 prior to expiration of the confirmation delay 428 in response to the atrial event sensing threshold crossing 414 by atrial EGM signal 402. Because a second sensed cardiac event signal is received during the confirmation delay 428, control circuit 206 determines the V/A sense ratio. Control circuit 206 may determine the V/A sense ratio as the ratio of the ventricular EGM maximum peak amplitude 438 to the atrial EGM maximum peak amplitude 418. The maximum peak amplitudes 418 and 438 may be determined from the respective atrial and ventricular EGM signals sensed during the confirmation delay 428 or within a post-sense time interval, e.g., up to 100 ms after the respective sensing threshold crossing 414 or 434.

Control circuit 206 may compare the V/A sense ratio (or other quantitative relationship) to an R-wave confirmation threshold because the Vsense event signal 446 is the first sensed event signal outside a confirmation delay and the Asense event signal 448 is received within the confirmation delay 428. In this case, the ratio of the maximum peak amplitude 438 of the true R-wave 436 to the maximum peak amplitude 418 of the far-field R-wave 416 in the atrial EGM signal 402 is relatively high. Control circuit 206 may confirm the Vsense event signal 446 as corresponding to a true R-wave 436 in response to the V/A sense ratio being greater than the R-wave confirmation threshold. The Asense signal 448 may be identified as a CCOS event signal, corresponding to an oversense far-field R-wave 416 on the atrial sensing channel 203. In some examples, control circuit 206 may additionally or alternatively compare the V/A sense ratio (or other quantitative relationship) to a P-wave confirmation threshold. When the V/A sense ratio is greater than the P-wave confirmation threshold and/or the V/A sense ratio is greater than the R-wave confirmation threshold, the Vsense event signal 446 is confirmed as a true sensed R-wave. Control circuit 206 may perform one or more pacing control and/or sensing control responses to the confirmation of the sensed R-wave.

In other examples, control circuit 206 may compare the V/A sense ratio to a previously established P-wave confirmation threshold and an R-wave confirmation threshold. Control circuit 206 may determine which of the P-wave confirmation threshold and the R-wave confirmation threshold the V/A sense ratio (or other quantitative relationship) is closest to. Control circuit 206 may confirm a true P-wave when the V/A sense ratio is closest to the P-wave confirmation threshold. Control circuit 206 may confirm a true R-wave when the V/A sense ratio is closest to the R-wave confirmation threshold.

In other examples, control circuit 206 may determine the quantitative relationship as a difference between the ventricular EGM maximum peak amplitude and the atrial EGM maximum peak amplitude. The difference may be determined as an absolute value or may be determined as a positive or negative value. The difference may be determined as the ventricular EGM maximum peak amplitude 438 minus the atrial EGM maximum peak amplitude 418 or vice versa. The difference may be compared to an R-wave confirmation threshold and/or a P-wave confirmation threshold. For example, when the difference determined as the ventricular EGM maximum peak amplitude minus the atrial EGM maximum peak amplitude, if the difference is greater than an R-wave confirmation threshold or closer to the R-wave confirmation threshold, an R-wave may be confirmed. If the difference is less than a P-wave confirmation threshold or closer to the P-wave confirmation threshold, a P-wave may be confirmed. In still other examples, the quantitative relationship may be determined as a sum or a product of the ventricular EGM maximum peak amplitude and the atrial EGM maximum peak amplitude. The sum or product may be compared to a respective R-wave confirmation threshold and/or P-wave confirmation threshold.

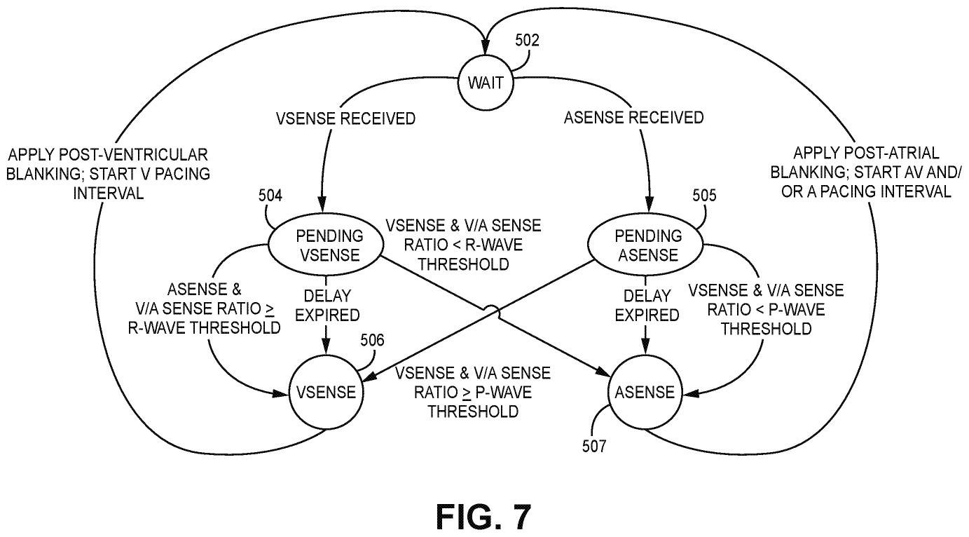

FIG. 7 is a state diagram 500 illustrating sensing operating states of pacemaker 14 according to some examples. At block 502, control circuit 206 may be in a pending state waiting to receive a cardiac event signal. As described above, control circuit 206 may receive an Asense event signal or a Vsense event signal from sensing circuit 204. In other examples, control circuit 206 may receive the cardiac event signal by receiving the atrial EGM signal and detecting an atrial event signal from the atrial EGM signal, e.g., based on a P-wave sensing threshold, waveform morphology or other criteria, or by receiving the ventricular EGM signal and detecting a ventricular event signal from the ventricular EGM signal, e.g., based on an R-wave sensing threshold, waveform morphology or other criteria. For the sake of illustration, in the description of sensing state diagram 500 and other flow charts presented herein, control circuit 206 is described as receiving an atrial event signal as an Asense signal and a ventricular event signal as a Vsense signal from respective atrial and ventricular sensing channels of sensing circuit 204.

In response to a Vsense signal being received outside a confirmation delay, control circuit 206 may transition from the pending, wait state 502 to a pending Vsense state 504 and start the confirmation delay. Control circuit 206 may transition to a confirmed Vsense state 506 from the pending Vsense state 504 in response to the confirmation delay expiring without an Asense event signal (center arrow 516). In other instances, control circuit 206 may transition to the confirmed Vsense state 506 from the pending Vsense state 504 in response to receiving an Asense event signal before the confirmation delay expires when the V/A sense ratio (or another quantitative relationship such as a V-A difference or a V-A sum or V-A product) is greater than an R-wave confirmation threshold (left arrow 514). Control circuit 206 may transition from the pending Vsense state 504 to a confirmed Asense state 507 in response to receiving an Asense event signal before the confirmation delay expires and the V/A sense ratio (or other quantitative relationship) being less than the R-wave confirmation threshold (right arrow 518).

In FIG. 7, a transition to the Vsense state 506 occurs in response to the V/A sense ratio being equal to the R-wave confirmation threshold (when an Asense follows a Vsense) or equal to the P-wave threshold (when a Vsense follows an Asense). It is to be understood that in other examples a transition to the Asense state 507 may occur in response to the V/A sense ratio being equal to the R-wave confirmation threshold or being equal to the P-wave confirmation threshold. The response to the V/A sense ratio being equal to one of the P-wave confirmation threshold or the R-wave confirmation threshold may depend on the values selected or programmed as the P-wave confirmation threshold or the R-wave confirmation threshold selected.

In response to transitioning to the confirmed Vsense state 506, control circuit 206 may apply post-ventricular blanking periods and transition back to the wait state 502. For example, control circuit 206 may pass a confirmed Vsense signal back to sensing circuit 204. Ventricular sensing channel 205 may apply a post-sense ventricular blanking period to the ventricular EGM signal starting from the time of the Vsense event signal. Atrial sensing channel 203 may apply a post-ventricular atrial blanking period to the atrial EGM signal starting from the time of the Vsense event signal.

When control circuit 206 receives an Asense event signal during the wait state 502, control circuit 206 may transition to the pending Asense state 505. Control circuit 206 may start the confirmation delay. Control circuit 206 may transition to the confirmed Asense state 507 in response to the confirmation delay expiring without a Vsense signal being received (center arrow 517) or in response to a Vsense signal being received during the confirmation delay and the V/A sense ratio (or another quantitative relationship such as the V-A difference, V-A sum or V-A product) being less than (or equal to) the P-wave confirmation threshold (right arrow 515). Control circuit 206 may transition from the pending Asense state 505 to the confirmed Vsense state 506 in response to a Vsense signal being received during the confirmation delay and the V/A sense ratio (or other quantitative relationship) being greater than the P-wave confirmation threshold (left arrow 519).

In response to transitioning to the confirmed Asense state 507, control circuit 206 may apply post-atrial blanking periods and transition back to the wait state 502. For example, control circuit 206 may pass a confirmed Asense signal back to sensing circuit 204. Atrial sensing channel 203 may apply a post-sense atrial blanking period to the atrial EGM signal starting from the time of the Asense event signal. Ventricular sensing channel 205 may apply a post-atrial ventricular blanking period to the ventricular EGM signal starting from the time of the Asense event signal. The blanking periods applied in response to a confirmed Vsense signal or a confirmed Asense signal cause sensing circuit 204 to ignore or discard any sensing threshold crossings that may occur by the atrial or ventricular EGM signal during a blanking period applied to the respective signal.

In addition to applying blanking periods according to the confirmed cardiac event signal, control circuit 206 may start a pacing interval in accordance with the current pacing mode and the confirmed cardiac event signal. In response to transitioning to the confirmed Asense state 507, control circuit 206 may start an AV pacing interval (during an atrial synchronous ventricular pacing mode) and/or an atrial pacing interval, e.g., an atrial LRI or rate response pacing interval (during a single chamber atrial pacing mode or during a dual chamber pacing mode). A scheduled, pending atrial pacing pulse may be inhibited upon transitioning to the Asense state 507.

In response to transitioning to the confirmed Vsense state 506, control circuit 206 may start a ventricular pacing interval. A scheduled, pending ventricular pacing pulse may be inhibited upon transitioning to the Vsense state 506. The pacing interval(s) started in response to a confirmed Vsense or Asense event signal may be started to have an effective starting time at the time of the received Vsense or Asense event signal, respectively. In other examples, any delay in a scheduled pacing pulse due to the confirmation delay and processing time to transition from the pending Vsense state 504 or Asense state 505 to a confirmed state 506 or 507 may be considered negligible or the pacing intervals may be adjusted by pace timing circuit 242 to account for the confirmation delay and processing time. In various examples, any blanking period(s), refractory period(s), pacing escape interval(s) or other timing intervals started by control circuit 206 in response to a confirmed cardiac event signal may be adjusted to have an effective starting time corresponding to the time of the received Asense or Vsense event signal that is the confirmed cardiac event signal.

In some examples, the confirmation delay is a programmable value that may be a single value that is started as a time interval in response to an Asense event signal or a Vsense event signal, whichever is received first. As noted previously herein, in other examples the confirmation delay may be a first confirmation delay or a second confirmation delay different than the first confirmation delay, depending on whether the Asense event signal is received first or the Vsense event signal is received first. A longer or shorter confirmation delay may be started by control circuit 206 in response to an Asense event signal than the confirmation delay started by control circuit 206 in response to a Vsense event signal. It is further noted that in some examples the confirmation delay may be programmable by a clinician or other user to be 0 ms. In some patients, CCOS may not arise (or may seldom occur) such that application of a confirmation delay is not necessary. When the confirmation delay(s) is (are) programmed to 0 ms, Asense event signals and Vsense event signals received by control circuit 206 are responded to as true, confirmed Asense event signals and Vsense event signals, respectively, for controlling sensing and/or pacing responses to the received cardiac sensed event signals.

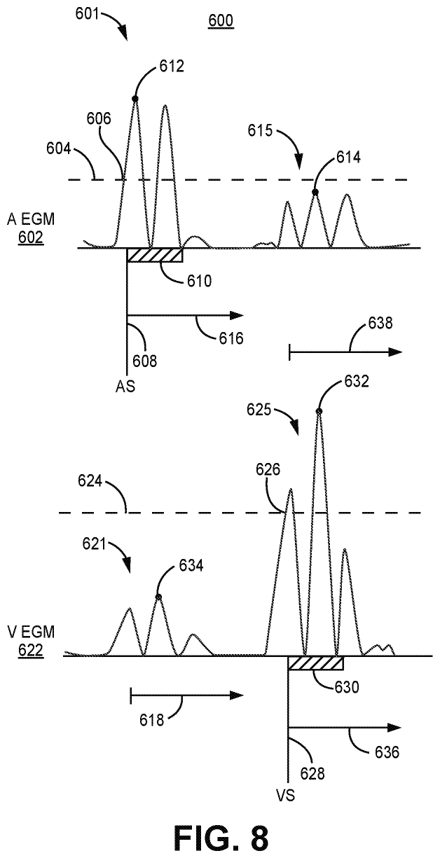

FIG. 8 is a diagram 600 of an atrial EGM signal 602 and a ventricular EGM signal 622 that may be sensed by sensing circuit 204. Each of the atrial EGM signal 602 and ventricular EGM signal 604 can be filtered, amplified and rectified EGM signals that can be passed to atrial event detector 226 or ventricular event detector 256, respectively, and/or passed to control circuit 206 for identifying a respective P-wave signal or R-wave signal. Rectified atrial EGM signal 602 includes a true P-wave 601 and a far-field R-wave wave 615. Rectified ventricular EGM signal 622 includes a true R-wave 625 and a cross channel P-wave 621.

Atrial sensing channel 203 may produce an Asense event signal 608 in response to atrial EGM signal 602 crossing the P-wave sensing threshold 604 at threshold crossing 606. Control circuit 206 may start the confirmation delay 610 in response to receiving Asense event signal 608 (when a confirmation delay is not already in progress). In this example, the cross-chamber P-wave 621 is not sensed during the confirmation delay 610, and the following R-wave 625 is sensed after the expiration of confirmation delay 610. As such, control circuit 206 may confirm the Asense event signal 608 as being a true, sensed P-wave.

In some examples, when a P-wave is confirmed based on the confirmation delay 610 expiring without a Vsense event signal, control circuit 206 may determine a V/A sense ratio (or other quantitative relationship) representative of a true, P-wave when no cross-chamber oversensing is occurring. While FIG. 8 is described with reference to the quantitative relationship being a V/A sense ratio, it is to be understood that the techniques described in conjunction with FIG. 8 (and other flow charts and diagrams presented herein) may be performed using a different quantitative relationship (e.g., difference, sum or product) of the ventricular and atrial EGM signal features than the sense ratio described here. The atrial EGM maximum peak amplitude 612 and the ventricular EGM maximum peak amplitude 634 following the Asense event signal 608 may be determined. The maximum peak amplitudes 612 and 634 may be determined from the respective atrial EGM signal 602 and ventricular EGM signal 622 sensed during the confirmation delay 610 or during a longer post-sense time interval, e.g., within 20 to 100 ms after the Asense event signal 608. The V/A sense ratio (or other quantitative relationship) determined from peak amplitudes 612 and 634 when a P-wave is confirmed without a CCOS event can be stored in memory 210 and/or used by control circuit 206 for establishing or updating the P-wave confirmation threshold applied to V/A sense ratios (or other quantitative relationships) determined when a pending Asense event signal is followed by a Vsense event signal within the confirmation delay during a future cardiac cycle.

In some examples, the V/A sense ratio determined as the ratio of the ventricular EGM maximum peak amplitude 634 to the atrial EGM maximum peak amplitude 612 is buffered in memory with multiple V/A sense ratios determined when an Asense event signal is confirmed based on an expired confirmation delay without a Vsense event signal. The P-wave confirmation threshold may be determined as a representative value of the multiple buffered V/A sense ratios, e.g., a mean, median, trimmed mean, trimmed median, maximum or other representative value. In other examples, a single V/A sense ratio may be used by control circuit 206 to update or establish a P-wave confirmation threshold. Example techniques for establishing or updating a P-wave confirmation threshold applied to a V/A sense ratio are described below in conjunction with FIG. 9.

In response to confirming the Asense event signal 608, sensing circuit 204 may apply a post-sense atrial blanking period 616 to the atrial EGM signal 602. The post-sense atrial blanking period 616 may have an effective starting time at the time of the Asense event signal 608. Any P-wave sensing threshold crossing during the post-sense atrial blanking period 616 is not sensed as a P-wave, does not result in another Asense event signal being produced by atrial sensing channel 203, or is ignored by control circuit 206 in controlling atrial and ventricular pacing.

In response to confirming the Asense event signal 608, sensing circuit 204 may apply a post-atrial ventricular blanking period 618 to the ventricular EGM signal 622. The post-atrial ventricular blanking period 618 may have an effective starting time at the time of the Asense event signal 608. Any subsequent R-wave sensing threshold crossing during the post-atrial ventricular blanking period 618 is not sensed as an R-wave, does not result in a Vsense event signal being produced by ventricular sensing channel 205, or is ignored by control circuit 206 in controlling ventricular pacing. The post-atrial blanking period 616 and the post-atrial ventricular blanking period 618 may be longer than the confirmation delay 610 as shown in FIG. 8 but are not necessarily longer than the confirmation delay 610.

Ventricular sensing channel 205 may produce a Vsense event signal 628 in response to ventricular EGM signal 622 crossing the R-wave sensing threshold 624 at threshold crossing 626. Control circuit 206 may start the confirmation delay 630 in response to receiving Vsense event signal 628 (if a confirmation delay is not already in progress). In this example, the far-field R-wave 615 in the atrial EGM signal 602 is not sensed by atrial sensing circuit 203 during the confirmation delay 630. As such, control circuit 206 may confirm the Vsense event signal 628 as being a true, sensed R-wave upon expiration of confirmation delay 630.

In some examples, when an R-wave is confirmed based on the confirmation delay 630 expiring without an Asense event signal, control circuit 206 may determine a V/A sense ratio (or other quantitative relationship) representative of a true, R-wave when no cross-chamber oversensing is occurring. The atrial EGM maximum peak amplitude 614 and the ventricular EGM maximum peak amplitude 632 following the Vsense event signal 628 may be determined. The maximum peak amplitudes 614 and 632 may be determined from the respective atrial EGM signal 602 and ventricular EGM 622 sensed during the confirmation delay 630 or during a longer post-sense time interval, e.g., within 20 to 100 ms of the Vsense signal 628. The V/A sense ratio (or other quantitative relationship) determined when an R-wave is confirmed without a CCOS event can be stored in memory 210 and/or used by control circuit 206 for establishing or updating the R-wave confirmation threshold applied to V/A sense ratios (or other quantitative relationships) determined when a pending Vsense event signal is followed by an Asense event signal within the confirmation delay during a future cardiac cycle.

In some examples, the V/A sense ratio determined as the ratio of ventricular EGM maximum peak amplitude 632 to atrial EGM maximum peak amplitude 614 is buffered in memory with multiple V/A sense ratios determined when a Vsense signal is confirmed to be a true R-wave based on an expired confirmation delay without an Asense event signal. The R-wave confirmation threshold may be determined as a representative value of the multiple buffered V/A sense ratios, e.g., a mean, median, trimmed mean, trimmed median, minimum or other representative value. In other examples, a single V/A sense ratio may be used by control circuit 206 to update or establish an R-wave confirmation threshold. Example techniques for establishing or updating an R-wave confirmation threshold applied to a V/A sense ratio are described below in conjunction with FIG. 9. As described above, in other examples the quantitative relationship between the atrial EGM signal feature and the ventricular EGM signal feature may be determined as a difference, sum, product or any combination thereof instead of a ratio.

In response to confirming the Vsense event signal 628, sensing circuit 204 may apply a post-sense ventricular blanking period 636 to the ventricular EGM signal 622. The post-sense ventricular blanking period 636 may have an effective starting time at the time of the Vsense event signal 628. Any subsequent R-wave sensing threshold crossing during the post-sense ventricular blanking period 636 is not sensed as an R-wave by sensing circuit 204, does not result in another Vsense event signal being produced by ventricular sensing channel 205, or is ignored by control circuit 206 in controlling ventricular pacing pulses.

In response to confirming the Vsense event signal 628, sensing circuit 204 may apply a post-ventricular atrial blanking period 638 to atrial EGM signal 602. The post-ventricular atrial blanking period 638 may have an effective starting time at the time of the Vsense event signal 628. Any subsequent P-wave sensing threshold crossing during the post-ventricular atrial blanking period 638 is not sensed as a P-wave, does not result in an Asense event signal being produced by atrial sensing channel 203, or is ignored by control circuit 206 in controlling atrial and ventricular pacing pulses. The post-sense ventricular blanking period 636 and the post-ventricular atrial blanking period 638 may be longer than the confirmation delay 630 as shown in FIG. 8 but are not necessarily longer than the confirmation delay 630.

While not shown in FIG. 8, but as generally described above, control circuit 206 may start one or more pacing intervals, e.g., an AV pacing interval, atrial LRI, ventricular LRI, rate response interval, or rate smoothing interval, in response to a confirmed cardiac event signal. A pending pacing pulse scheduled for delivery upon expiration of a pacing interval that is running at the time of a confirmed cardiac event signal, P-wave or R-wave, may be inhibited in accordance with the current pacing mode in effect. In other examples, a pacing pulse may be triggered in response to a confirmed P-wave or a confirmed R-wave, e.g., for providing atrial or ventricular safety pacing or starting an AV pacing interval.

When a ventricular pacing escape interval expires during the confirmation delay 610 after the Asense event signal 608, the pending ventricular pacing pulse may be delivered when the pacing escape interval expires before a Vsense event signal is received after the Asense event signal 608. The confirmation delay 610 started in response to the Asense event signal 608 may be cancelled (e.g., terminated without further analysis of the atrial and ventricular EGM signals). In other examples, the pending ventricular pacing pulse may be delayed or withheld until the expiration of the confirmation delay 610. If the Asense event signal 608 is confirmed (based on no Vsense event signal during the confirmation delay 610 or based on analysis of the quantitative relationship of the atrial and ventricular EGM signal features), the pending ventricular pacing pulse may be delivered upon expiration of the confirmation delay 610. If a Vsense event signal is received during the confirmation delay 610 and the Vsense event signal is confirmed based on the V/A sense ratio or other quantitative relationship, the pending ventricular pacing pulse can be inhibited.

When an atrial pacing escape interval expires after the Vsense event signal 628 during the confirmation delay 630, the pending atrial pacing pulse may be delivered when the atrial pacing escape interval expires before an Asense event signal is received after the Vsense event signal 628. The confirmation delay 630 started in response to the Vsense event signal 628 may be cancelled (e.g., terminated without further analysis of the atrial and ventricular EGM signals). In other examples, the pending atrial pacing pulse may be delayed or withheld until the expiration of the confirmation delay 630. If the Vsense event signal 628 is confirmed, the pending atrial pacing pulse may be delivered upon expiration of the confirmation delay 630. If an Asense event signal is received during the confirmation delay 630 and the Asense event signal is subsequently confirmed based on the V/A sense ratio (or other quantitative relationship), the pending atrial pacing pulse can be inhibited (e.g., cancelled).

FIG. 9 is a flow chart 700 of a method that may be performed by pacemaker 14 for establishing or updating P-wave and/or R-wave confirmation thresholds according to some examples. Control circuit 206 may be configured to establish and/or adjust a P-wave and/or R-wave confirmation threshold based on analysis of the atrial EGM signal and/or the ventricular EGM signal. Analysis of the atrial EGM signal and/or ventricular EGM signal may be performed to determine one or more quantitative relationship(s) between the atrial EGM signal and ventricular EGM signal when CCOS is not occurring and/or when CCOS is occurring. The quantitative relationship(s) may be determined and used by control circuit 206 to set or adjust the P-wave confirmation and/or R-wave confirmation threshold(s).

In the example method of FIG. 9, at block 702, control circuit 206 may receive an atrial event signal as an Asense event signal from sensing circuit 204, produced by atrial sensing channel 204 in response to a P-wave sensing threshold crossing by the atrial EGM signal. As described above, in other examples, control circuit 206 may receive an atrial event signal by receiving the atrial EGM signal from sensing circuit 204 and identifying a P-wave from the atrial event signal based on a threshold crossing, waveform morphology or other waveform features of the atrial EGM signal.

In some instances, an atrial pacing pulse (Apace) may be delivered when an atrial pacing escape interval expires before receiving an Asense event signal. In this case, control circuit 206 may advance to block 710 to wait for a Vsense signal. In response to receiving an Asense event signal at block 702, control circuit 206 may start the confirmation delay at block 704. If a Vsense event signal is received at block 706 (or an R-wave is identified by control circuit 206 from a received ventricular EGM signal) before the confirmation delay expires, control circuit 206 may ignore the Asense event signal for the purposes of establishing or updating a P-wave confirmation threshold. Control circuit 206 may return to block 702 to wait for the next Asense signal received outside of a confirmation delay.

In other examples, control circuit 206 may advance to block 710 to wait for a Vsense event signal received outside a confirmation delay. While not shown in FIG. 9, it is to be understood that in response to receiving the Vsense signal at block 706 during the confirmation delay following the Asense signal at block 702, control circuit 206 may determine the V/A sense ratio for confirming one of the Asense or the Vsense event signals based on a current value of the P-wave confirmation threshold (and/or an R-wave confirmation threshold) according to any of the examples described above.

When a Vsense event signal is not received during the confirmation delay following an Asense event signal (“no” branch of block 706), control circuit 206 may determine the V/A sense ratio at block 708 (or a V-A difference), as generally described above in conjunction with FIG. 8. For example, with reference to FIG. 8, control circuit 206 may determine the ratio of ventricular EGM maximum peak amplitude 634 to atrial EGM maximum peak amplitude 612 as the V/A sense ratio at block 708. Control circuit 206 may use the determined V/A sense ratio to establish or update a P-wave confirmation threshold, as further described below. In this way, the V/A sense ratio (or other quantitative relationship determined at block 708) can be referred to as a “reference quantitative relationship” because it is determined by control circuit 206 when the Asense event signal is received without a Vsense event signal being received within the confirmation delay. The reference quantitative relationship is therefore a relationship of the atrial EGM signal feature and the ventricular EGM signal feature when CCOS is not occurring. This reference quantitative relationship can be used for setting or adjusting the P-wave confirmation threshold that is subsequently applied to the V/A sense ratio when an Asense event signal and a Vsense event signal do occur within a confirmation delay of each other.

Referring again to FIG. 9, control circuit 206 may wait for a Vsense event signal received from sensing circuit 204 outside a confirmation delay at block 710. In other examples, control circuit 206 may receive a ventricular event signal by receiving the ventricular EGM signal from sensing circuit 204 and identifying an R-wave based on a threshold crossing, waveform morphology or other waveform features. If a ventricular pacing pulse (Vpace) occurs before a Vsense event signal is received outside of a confirmation delay, control circuit 206 may return to block 702 to wait for the next Asense signal. When a Vsense event signal is received at block 710 outside of a confirmation delay, control circuit 206 may start the confirmation delay at block 712 in response to the received Vsense event signal.

If an Asense event signal is received at block 714 before the confirmation delay expires (“yes” branch of block 714), control circuit 206 may return to block 710 to wait for another Vsense event signal received outside a confirmation delay. In other examples, as shown in FIG. 9, control circuit 206 may return to block 702 to wait for an Asense event signal received outside a confirmation delay. In general, control circuit 206 may wait for the next sensed cardiac event signal, atrial or ventricular, that occurs outside a running confirmation delay for acquiring V/A sense ratios (or other quantitative relationship) from the atrial and ventricular EGM signals when Asense event signals are received without a Vsense event signal within the confirmation delay and/or when Vsense event signals are received without an Asense event signal within the confirmation delay. The P-wave and R-wave confirmation thresholds may not be updated based on EGM signals sensed when both an Asense event signal and a Vsense event signal occur within the confirmation delay from each other. However, in this case, control circuit 206 may confirm one of the Asense event signal or the Vsense event signal using the techniques disclosed herein based on current or nominal values of the P-wave confirmation threshold and/or R-wave confirmation threshold.

When an Asense event signal is not received before expiration of the confirmation delay following a Vsense event signal (“no” branch of block 714), control circuit 206 may determine the V/A sense ratio (or a V-A difference or other quantitative relationship) at block 716. The V/A sense ratio may be determined as generally described above in conjunction with FIG. 8. For example, with reference to FIG. 8, control circuit 206 may determine the ratio of ventricular EGM maximum peak amplitude 632 to atrial EGM maximum peak amplitude 614 as the V/A sense ratio at block 716. Control circuit 206 may use the determined V/A sense ratio to establish or update an R-wave confirmation threshold at block 718. The V/A sense ratio (or other quantitative relationship) determined at block 716 by control circuit 206 when the Vsense event signal is received without an Asense event signal being received within the confirmation delay can be referred to as a “reference quantitative relationship” of the atrial EGM signal feature and the ventricular EGM signal feature when CCOS is not occurring. As described below, this reference quantitative relationship can be used for setting or adjusting the R-wave confirmation threshold.

Using the V/A sense ratio determined at block 708 for an Asense event signal that is confirmed without a Vsense event signal during the confirmation delay, control circuit 206 may establish or update a P-wave confirmation threshold at block 718. The P-wave confirmation threshold may be set to a percentage of one V/A sense ratio or the mean, median, maximum or other representative value of multiple V/A sense ratios determined for one or more confirmed P-waves in the absence of cross-chamber oversensing. The percentage may be 100%, 110%, 120%, 150% or other selected percentage. The P-wave confirmation threshold may be set to a percentage of a representative V/A sense ratio plus an offset in some examples. The offset may be positive 0.1, 0.2, 0.3, 0.5, 1.0 or other selected offset. The offset may be scaled based on the atrial EGM maximum peak amplitude, the ventricular EGM maximum peak amplitude or the currently determined V/A sense ratio for the Asense event signal determined or received at block 702. The P-wave confirmation threshold may be set to be greater than all V/A sense ratios determined for one or more P-waves that are confirmed by control circuit 206 based on no Vsense event signal being received during the confirmation delay after the Asense event signal is received.

A previously established P-wave confirmation threshold may be updated based on the V/A sense ratio determined at block 708. In some examples, the P-wave confirmation threshold may be an initial, user-programmed or default value, e.g., 0.5, 0.7, or 1.0 as illustrative examples. The P-wave confirmation threshold may be updated at block 718 by adjusting the P-wave confirmation threshold by an increment or decrement toward the V/A sense ratio determined at block 708 (e.g., ±0.05, 0.1, 0.2 or other increment or decrement). In other examples, the P-wave confirmation threshold may be updated to be greater than the V/A sense ratio determined at block 708. Control circuit 206 may not adjust the P-wave confirmation threshold to be greater than the R-wave confirmation threshold.

Using the V/A sense ratio determined at block 716 for a Vsense event signal that is confirmed without an Asense event signal during the confirmation delay, control circuit 206 may establish or update an R-wave confirmation threshold at block 718. The R-wave confirmation threshold may be set to a percentage of one V/A sense ratio or the mean, median, minimum or other representative value of multiple V/A sense ratios determined for one or more confirmed R-waves in the absence of cross-chamber oversensing. The percentage may be 60%, 70%, 80%, 90% or any other selected percentage. The R-wave confirmation threshold may be set to a percentage of a representative V/A sense ratio minus an offset in some examples. The offset may be 0.1, 0.2, 0.3, 0.5, 1.0 or other selected offset. The offset may be scaled based on the atrial EGM maximum peak amplitude, the ventricular EGM maximum peak amplitude or the V/A sense ratio determined for the current Vsense event signal received at block 710. The offset may be scaled based on a representative V/A sense ratio determined from multiple, buffered V/A sense ratios determined from confirmed R-waves without CCOS. The R-wave confirmation threshold may be set to be less than all V/A sense ratios determined for one or more R-waves that are confirmed by control circuit 206 based on no Asense event signal being received during the confirmation delay after a Vsense event signal is received.

A previously established R-wave confirmation threshold may be updated at block 718 based on the V/A sense ratio determined at block 716. In some examples, the R-wave confirmation threshold may be an initial, user-programmed or default value, e.g., 1.5, 2.0 or 2.5 as illustrative examples. The R-wave confirmation threshold may be updated at block 718 by adjusting the R-wave confirmation threshold by an increment or decrement toward the V/A sense ratio determined at block 716 (e.g., ±0.1, 0.2, 0.5 or other increment or decrement). In other examples, the R-wave confirmation threshold may be updated at block 718 to be less than the V/A sense ratio determined at block 716. Control circuit 206 may not adjust the R-wave confirmation threshold to be less than the P-wave confirmation threshold.

In some examples, control circuit 206 establishes or updates a P-wave confirmation threshold and an R-wave confirmation threshold at block 718 to be two distinct values. In other examples, control circuit 206 may establish one confirmation threshold that is applied to the V/A sense ratio for confirming P-waves and R-waves. A common P-wave confirmation threshold value and R-wave confirmation threshold value may be determined to be between a V/A sense ratio determined at block 708 and a V/A sense ratio determined at block 716. In some examples, control circuit 206 may buffer multiple V/A sense ratios for confirmed P-waves without CCOS and multiple V/A sense ratios for confirmed R-waves without CCOS sensing. The common P-wave confirmation threshold and R-wave confirmation threshold may be determined to be a mean of buffered V/A sense ratios or another value that is intermediate to the V/A sense ratios for confirmed P-waves and the V/A sense ratios for confirmed R-waves. In this case, the P-wave confirmation threshold and the R-wave confirmation threshold may be equal. As described above, it is to be understood that the quantitative relationship between an atrial EGM signal feature and a ventricular EGM signal feature may be determined as a difference, e.g., a V-A (or A-V) difference, instead of a ratio as described herein, for performing a comparative analysis of two signals sensed within the confirmation delay of each other.

For the sake of illustration, flow chart 700 shows determining V/A sense ratios at block 708 when a Asense event signal is received and determining V/A sense ratios at block 716 when a Vsense event signal is received. It is to be understood that control circuit 206 may determine V/A sense ratios only at block 708 or only at block 716. V/A sense ratios determined for both Asense event signals (without Vsense event signals within the confirmation delay) and Vsense event signals (without Asense event signals within the confirmation delay) may not be required for establishing or updating the confirmation threshold(s) at block 718. The confirmation threshold(s) may be updated at block 718 based on V/A sense ratios determined from the atrial and ventricular EGM signals when either Asense event signals are received without Vsense event signals within the confirmation delay or when Vsense event signals are received without Asense event signals within the confirmation delay. A single confirmation threshold may be established or a distinct P-wave confirmation threshold and R-wave confirmation threshold may be established based on the V/A sense ratios determined at either block 708 or block 716, but not necessarily both. For example, the P-wave confirmation threshold may be set to be a portion of (less than) the V/A sense ratios determined at block 708, and the R-wave confirmation threshold may be set to be a multiple of (greater than) the V/A sense ratios determined at block 708. In another example, the P-wave confirmation threshold may be set to be a first percentage of the V/A sense ratios determined at block 716, and the R-wave confirmation threshold may be set to a second percentage greater than the first percentage of the V/A sense ratios determined at block 716. In other examples, a single confirmation threshold may be established based on V/A sense ratios determined at either block 708 or 716. When the V/A sense ratio determined for a subsequent Asense event signal and Vsense event signal received within the single confirmation delay from each other is less than the confirmation threshold, the Asense event signal can be confirmed as being a true P-wave by control circuit 206. The Vsense event signal can be identified as a CCOS event. When the V/A sense ratio determined for a subsequent Asense event signal and Vsense event signal received within the confirmation delay from each other is greater than the single confirmation threshold, the Asense event signal can be identified as a CCOS event (e.g., far field R-wave) by control circuit 206, and the Vsense event signal can be confirmed as a true sensed R-wave.

Control circuit 206 may perform the process of flow chart 700 upon pacemaker implantation, during a patient follow-up, on a periodic scheduled basis (e.g., once per minute, once per hour, once per day, etc.), or on a beat-by-beat basis, e.g., concurrently or in parallel with the techniques for confirming cardiac sensed event signals described above. In other examples, the process of flow chart 700 may additionally or alternatively be triggered to be performed when atrial sensitivity, ventricular sensitivity or another sensing control parameter used to sense cardiac event signals is programmed or adjusted to a new value. The process of flow chart 700 may be triggered to be performed in response to a pacing mode switch or when pacemaker performance is different than expected, e.g., when atrial pacing burden is higher than expected for the patient (e.g., greater than a programmed threshold atrial pacing burden), asynchronous ventricular pacing burden is higher than expected for the patient (e.g., greater than a programmed threshold ventricular pacing burden), when a percentage of atrial synchronous ventricular pacing pulses delivered by pacemaker 14 is less than a threshold percentage of all delivered ventricular pacing pulses or all ventricular electrical events (sensed and paced). In some examples, control circuit 206 may be configured to detect an arrhythmia, e.g., an atrial tachyarrhythmia, ventricular tachycardia, or ventricular fibrillation. For instance, control circuit 206 may detect a tachyarrhythmia based on the rate of atrial sensed event signals and/or ventricular sensed event signals. The process of flow chart 700 may be triggered to be performed when an arrhythmia has been or is being detected by control circuit 206.

FIG. 10 is a flow chart 750 of a method for establishing bandpass filter cutoff frequencies for sensing an atrial EGM signal and a ventricular EGM signal according to some examples. Identically numbered blocks in FIG. 10 generally correspond to like-numbered blocks described above in conjunction with FIG. 9. In the process of flow chart 750, control circuit 206 may adjust the bandpass cutoff frequencies of the atrial sensing channel 203 and/or the ventricular sensing channel 205 for maximizing a difference between the V/A sense ratios (or other quantitative relationship) determined when a true P-wave is sensed and/or when a true R-wave is sensed. The high and/or low cutoff frequencies of atrial sensing channel filter 224 (see FIG. 4) may be adjusted to maximize the P-wave amplitude and minimize the far-field R-wave amplitude in the atrial EGM signal. The high and/or low cutoff frequencies of ventricular sensing channel filter 254 (see FIG. 4) may be adjusted to maximize the R-wave amplitude and minimize the cross-chamber P-wave amplitude in the ventricular EGM signal.

As generally described above in conjunction with FIG. 9, control circuit 206 may determine a V/A sense ratio at block 708 when an Asense event signal is received (block 702) without a Vsense event signal being received during the confirmation delay (“no branch of block 706). Control circuit 206 may determine a V/A sense ratio at block 716 when a Vsense event signal is received (block 710) without an Asense event signal being received during the confirmation delay (“no branch of block 714).

Control circuit 206 may determine the V/A sense ratio difference at block 752 between the V/A sense ratio determined at block 708 for a true P-wave and the V/A sense ratio determined at block 716 for a true R-wave. In some examples, control circuit 206 may adjust the bandpass filter settings of atrial sensing channel 203 and/or ventricular sensing channel 205 one or more times and repeat the process of determining the V/A sense ratios at block 708 and 716 for multiple filter settings to enable a comparison of the V/A sense ratio differences determined for different combinations of filter cutoff frequencies. At block 754, control circuit 206 may determine if another filter setting remains to be evaluated. Control circuit 206 may advance (or return) to block 756 to adjust the high pass cutoff frequency and/or low pass cutoff frequency of atrial sensing channel filter 224 and/or the high pass frequency and/or low pass frequency of ventricular sensing channel filter 254 to repeat the process of flow chart 750 for determining the V/A sense ratio difference at block 752 for different filter settings.

In other examples, control circuit 206 may compare a determined V/A sense ratio difference to a threshold difference at block 754. If the V/A sense ratio difference is not greater than a threshold difference, control circuit 206 may adjust the low and/or high cutoff frequency of the atrial sensing channel filter 224 and/or of the ventricular sensing channel filter 254 at block 756 and repeat the process for determining the V/A sense ratio difference until the V/A sense ratio difference is greater than the threshold difference at block 754. The V/A sense ratio difference may be required to at least 0.2, 0.25, 0.3, 0.4, 0.5, 0.6, 0.7, 0.8, 1.0, 1.25, 1.5 or 2.0 as examples, with no limitation intended.

When the V/A sense ratio difference is determined for multiple filter settings, control circuit 206 may determine the combination of bandpass filter settings that results in the greatest V/A sense ratio difference at block 756. Control circuit 206 may set the cutoff frequencies of the atrial sensing channel filter 224 and the ventricular sensing channel filter 254 according to the filter settings resulting in the greatest V/A sense ratio difference. In other examples, control circuit 206 may set the filter settings at block 756 according to bandpass filter cutoff frequencies that resulted in the V/A sense ratio difference to be greater than the threshold difference at block 754.

In some examples, other criteria may be used by control circuit 206 in setting the bandpass filter settings at block 756. For example, control circuit 206 may identify the combination of bandpass filter settings that result in the greatest P-wave amplitude in the atrial EGM signal, the greatest R-wave amplitude in the ventricular EGM signal, and a V/A sense ratio difference determined at block 752 that is greater than the threshold difference.

Furthermore, a different quantitative relationship may be used in addition to or instead of the V/A sense ratio. Control circuit 206 may determine the quantitative ratio at blocks 708 and 716 as a difference, e.g., V-A difference or A-V difference. At block 752, control circuit 206 may determine the difference or ratio between the quantitative relationship determined at block 706 when only an Asense signal is received and the quantitative ratio determined at block 716 when only a Vsense signal is received. Control circuit 206 may adjust at least one of the atrial EGM signal low pass filter cutoff frequency, the atrial EGM signal high pass filter cutoff frequency, the ventricular EGM signal low pass filter cutoff frequency, and/or the ventricular EGM signal high pass filter cutoff frequency to maximize the difference between the two quantitative relationships determined at block 706 and 716.

In some examples, the process of flow chart 750 may include receiving user input. For example, control circuit 206 may transmit the atrial EGM signal and the ventricular EGM signal to external device 20 (see FIG. 1). The bandpass filter settings of atrial sensing channel filter 224 and ventricular sensing channel filter 254 may be adjusted according to multiple available settings as the EGM signals are displayed to a user on display unit 54. The EGM signals may be annotated with Asense event makers, Vsense event markers, maximum peak P-wave amplitude, maximum peak R-wave amplitude and/or V/A sense ratios. A user may select the filter settings, which may be reported by control circuit 206 or external device processor 52, that result in the greatest V/A sense ratio for true R-waves, lowest V/A sense ratio for true P-waves, greatest sense ratio difference, greatest P-wave peak amplitude, greatest R-wave peak amplitude, lowest far-field R-wave peak amplitude in the atrial EGM signal, lowest cross-channel P-wave peak amplitude in the ventricular EGM signal or any combination thereof.

After setting the filter bandpass cutoff frequencies for each of the atrial sensing channel 203 and the ventricular sensing channel 205 at block 756, control circuit 206 may establish the P-wave confirmation threshold and the R-wave confirmation threshold at block 718. The P-wave confirmation threshold and the R-wave confirmation threshold may be established based on V/A sense ratios determined with the bandpass filter cutoff frequencies set at block 756. In some examples, once the bandpass filter settings are set at block 756, control circuit 206 may establish the P-wave confirmation threshold as a mean, median or other representative value of the V/A sense ratio determined when Asense event signals are received without a Vsense event signal during the confirmation delay. Control circuit 206 may establish the R-wave confirmation threshold as a mean, median or other representative value of the V/A sense ratio determined when Vsense events signals are received without an Asense event signal during the confirmation delay.

In some examples, control circuit 206 may confirm a true P-wave when the V/A sense ratio is nearer to the P-wave confirmation threshold than the R-wave confirmation threshold. Control circuit 206 may confirm a true R wave when the V/A sense ratio is nearer to the R-wave confirmation threshold than the P-wave confirmation threshold. In other examples, as described above, a true P-wave can be confirmed by control circuit 206 when the V/A sense ratio is less than the P-wave confirmation threshold. A true R-wave can be confirmed when the V/A sense ratio is greater than the R-wave confirmation threshold.

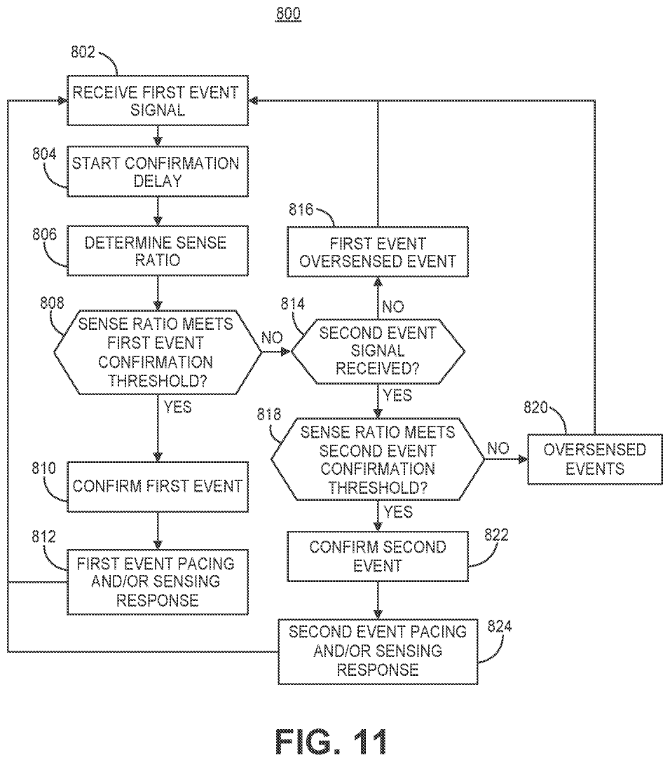

FIG. 11 is a flow chart 800 of a method that may be performed by pacemaker 14 for confirming sensed cardiac event signals according to another example. The techniques described above generally provide a method performed by a medical device for identifying a true cardiac event signal and a CCOS event signal when two cardiac event signals occur within the confirmation delay from each other. In some instances, one sensing channel of sensing circuit 204 may oversense an event signal when the other sensing channel undersenses the true event signal. In this case, a second event signal may not be received within the confirmation delay of an oversensed signal. In other instances, both sensing channels may oversense the same event signal that is not a true event signal intended to be sensed by either sensing channel. The process of flow chart 800 provides a technique for confirming a true event signal or identifying an oversensed event signal when one sensing channel oversenses an event signal and the other channel undersenses the event signal and/or when both channels oversense an event signal.

At block 802, control circuit 206 receives a first cardiac event signal, e.g., an Asense event signal or a Vsense event signal, when a confirmation delay is not already running. At block 804, control circuit 206 may start the confirmation delay in response to receiving the first cardiac event signal outside a confirmation delay (and outside any applicable blanking periods). At block 806, control circuit 206 may determine the V/A sense ratio (or another quantitative relationship) from the atrial EGM signal and the ventricular EGM signal sensed during the confirmation delay or during a specified post-sense time interval. In this example, the V/A sense ratio can be determined by control circuit 206 in response to the first sensed event signal regardless of whether a second sensed event signal is received during the confirmation delay.

At block 808, control circuit 206 may determine if the V/A sense ratio meets the confirmation threshold for confirming the first event signal. For example, if the first event signal is an Asense event signal, control circuit 206 may determine if the V/A sense ratio is less than the P-wave confirmation threshold at block 808. If the first event signal is a Vsense event signal, control circuit 206 may determine if the V/A sense ratio is greater than the R-wave confirmation threshold at block 808. When the V/A sense ratio meets the threshold requirement for confirming the first cardiac event signal received at block 802 (“yes” branch of block 808), control circuit 206 may confirm the first cardiac event signal at block 810. At block 812, control circuit 206 may perform a pacing and/or sensing response based on the confirmed first cardiac event signal and a currently operating pacing mode according to any of the example responses described herein. Control circuit 206 may return to block 802 to wait for the next cardiac event signal received outside a confirmation delay.

If the V/A sense ratio does not meet the first event confirmation threshold requirement at block 808 (“no” branch), control circuit 206 may determine if a second cardiac event signal (e.g., from the other sensing channel of sensing circuit 204) is received at block 814 before the confirmation delay expires. If a second event signal is not received, and the V/A sense ratio does not meet the first event confirmation threshold requirement at block 808, control circuit 206 may identify the first cardiac event signal as an oversensed event signal at block 816. The first cardiac event signal may be a CCOS event signal. For example, atrial sensing channel 203 may oversense a far-field R-wave when the same R-wave is undersensed (not sensed) by the ventricular sensing channel 204. In other instances, ventricular sensing channel 205 may oversense a P-wave that is undersensed by the atrial sensing channel 203. In another example, the ventricular sensing channel 205 may oversense a T-wave that is not sensed by atrial sensing channel 203. In still other examples, one sensing channel 203 or 205 may oversense a non-cardiac event signal, e.g., an electromagnetic interference (EMI) noise signal or other EGM noise contamination.

When the first cardiac event signal is identified as an oversensed event signal at block 816, control circuit 206 may return to block 802 to wait for the next cardiac event signal received outside a confirmation delay. Control circuit 206 may ignore the oversensed event signal for the purposes of providing a sensing and/or pacing response.

Referring again to block 814, if a second event signal is received during the confirmation delay (“yes” branch of block 814), control circuit 206 may optionally determine if the V/A sense ratio determined at block 806 meets the confirmation threshold requirement for confirming the second sensed event signal at block 818. For example, control circuit 206 may determine if the V/A sense ratio is greater than the R-wave confirmation threshold if the second cardiac event signal received during the confirmation delay is a Vsense event signal. Control circuit 206 may determine if the V/A sense ratio is less than the P-wave confirmation threshold if the second cardiac event signal received during the confirmation delay is an Asense event signal. If the V/A sense ratio meets the second cardiac event confirmation threshold requirement (“yes” branch of block 818), control circuit 206 may confirm the second cardiac event at block 822. In other examples, block 818 is optional. When a second cardiac event signal is received at block 814 before expiration of the confirmation delay and the V/A sense ratio does not meet the first cardiac event signal confirmation threshold at block 808, the second cardiac event signal may be confirmed at block 822.

In response to confirming the second cardiac event signal, control circuit 206 may perform one or more pacing and/or sensing responses at block 824 based on the confirmed second cardiac event signal and in accordance with the currently operating pacing mode. Control circuit 206 may return to block 802 to wait for the next cardiac event signal received outside of a confirmation delay.

When the V/A sense ratio determined at block 806 does not meet the first cardiac event confirmation threshold or the second cardiac event confirmation threshold (“no” branch of block 818), control circuit 206 may determine that both of the first cardiac event signal and the second cardiac event signal are oversensed event signals at block 820. In some instances, both the atrial sensing channel 203 and the ventricular sensing channel 205 may oversense a T-wave, an EMI signal or other noise signal. When the V/A sense ratio does not meet either of the P-wave confirmation threshold or the R-wave confirmation threshold, control circuit 206 may identify an Asense event signal and a Vsense event signal received within the confirmation delay from each other as oversensed event signals at block 820. In response to identifying oversensed event signals received from both of the atrial sensing channel 203 and the ventricular sensing channel 205, control circuit 206 may return to block 802. Control circuit 206 may ignore the oversensed event signals for the purposes of providing a sensing and/or pacing response, such as starting (or restarting) a pacing interval, inhibiting a pacing pulse, applying a blanking period, etc.

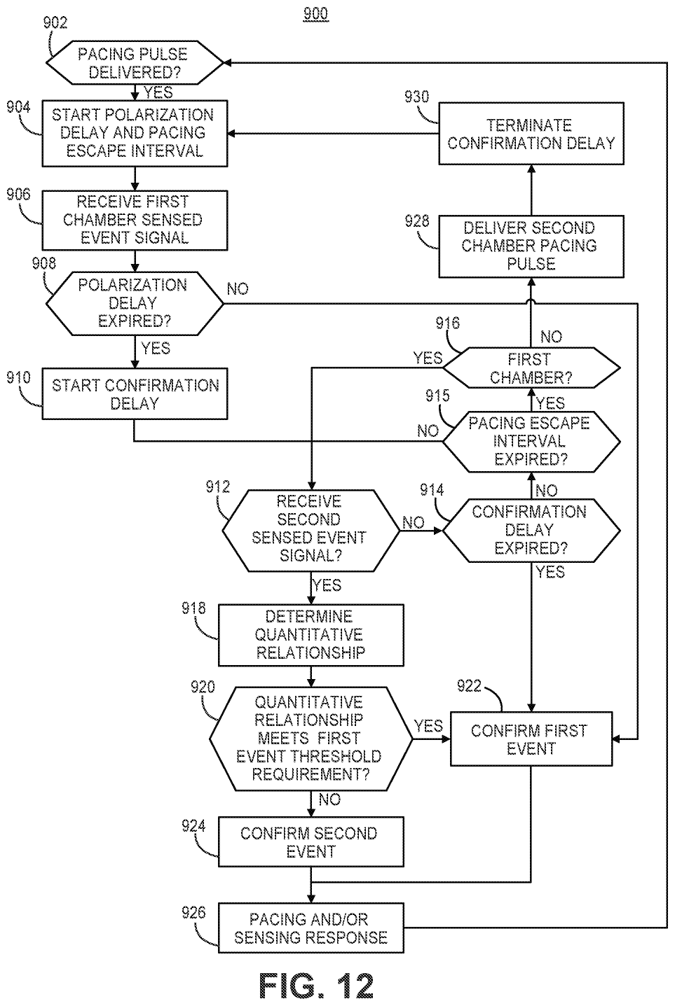

FIG. 12 is a flow chart 900 of a method for sensing cardiac event signals by a medical device and providing a pacing and/or sensing response to a sensed cardiac event signal according to another example. When a pacing pulse is delivered, post-pace polarization artifact may interfere with confirming a sensed event signal in some examples. Accordingly, in some examples, when a pacing pulse is delivered (block 902), control circuit 206 may optionally start a polarization delay at block 904. For example, a timer may be started by control circuit 206 to count down a polarization delay of 30 ms, 50 ms, 80 ms, 100 ms, 150 ms or other selected value for the polarization delay to allow any post-pace polarization artifact time to decay. It is to be understood that while waiting for a pacing pulse to be delivered at block 902, control circuit 206 may be receiving Asense event signals and/or Vsense event signals and performing the methods disclosed herein for confirming sensed cardiac event signals.

When a pacing pulse is delivered by pulse generator 202 at block 902, depending on the pacing mode, control circuit 206 may restart a pacing escape interval at block 904 in response to the delivered pacing pulse to schedule the next, pending pacing pulse. The pacing pulse delivered at block 902 may be an atrial pacing pulse such that an atrial pacing escape interval (e.g., an atrial LRI or a temporary rate response interval or rate smoothing interval) may be restarted at block 904. An AV pacing interval may be started at block 904 when an atrial pacing pulse is delivered at block 902 and the pacing mode is an atrial synchronous ventricular pacing mode. In other instances, the pacing pulse delivered at block 902 may be a ventricular pacing pulse such that a ventricular pacing escape interval (e.g., a ventricular LRI or a temporary rate response interval or rate smoothing interval) may be restarted at block 904 in accordance with the pacing mode of operation that is in effect.

When a sensed event signal is received by control circuit 206 at block 906 outside of a blanking period or a previously started confirmation delay, control circuit 206 may determine if the polarization delay is expired at block 908. If not, the first sensed event signal may be confirmed at block 922 without starting a confirmation delay. If the polarization delay is expired (“yes” branch of block 908), control circuit 206 may start the confirmation delay at block 910. As generally described above, if a second sensed event signal is not received (“no” branch of block 912) and the confirmation delay expires (“yes” branch of block 914), the first sensed event signal may be confirmed by control circuit 206 at block 922. If a second sensed event signal is received at block 912 (“yes” branch of block 912) prior to expiration of the confirmation delay, control circuit 206 may determine a quantitative relationship of an atrial EGM signal feature and a ventricular EGM signal feature at block 918 according to any of the examples described above. Control circuit 206 compares the quantitative relationship to a confirmation threshold at block 920 for confirming either the first sensed event signal (at block 922) or the second sensed event signal (at block 924) according to any of the example methods provided herein.

Control circuit 206 may control pulse generator 202 to provide a pacing response and/or control sensing circuit 204 to provide a sensing response according to the confirmed sensed event signal at block 926. Any of the example pacing and/or sensing responses described herein may be performed at block 926 based on which of the first or second sensed event signals is confirmed.

In some instances, a pacing escape interval may expire during the confirmation delay that is started at block 910. Referring again to block 914, if a confirmation delay is started and has not expired (“no” branch of block 914) and a pacing escape interval expires during the confirmation delay (“yes” branch of block 915), control circuit 206 may respond to the expired pacing escape interval based on which heart chamber the pending pacing pulse is scheduled to be delivered and/or which sensing channel, atrial or ventricular, generated the first sensed event signal at block 906.

For instance, if an Asense event signal is received at block 906 as the first heart chamber sensed event signal and a ventricular pacing escape interval expires during the confirmation delay, the scheduled, pending ventricular pacing pulse (for delivery to the second heart chamber) may be delivered at block 928 (“no” branch of block 916). When a Vsense event signal has not been received during the confirmation delay (“no” branch of block 912) and a ventricular pacing escape interval expires after an Asense event signal triggers the start of the confirmation delay, the ventricular pacing pulse may be delivered at block 928 in response to an expired ventricular pacing escape interval (which may be an AV delay, a ventricular LRI, rate response interval, rate smoothing interval, etc.). The confirmation delay may be terminated at block 930. The Asense event signal may be confirmed such that any post-atrial blanking and refractory periods and/or atrial pacing escape intervals may be started having an effective starting time at the time of the P-wave sensing threshold crossing, for example. Control circuit 206 may return to block 904 to start the polarization delay and restart a ventricular pacing escape interval as needed according to the operating pacing mode.

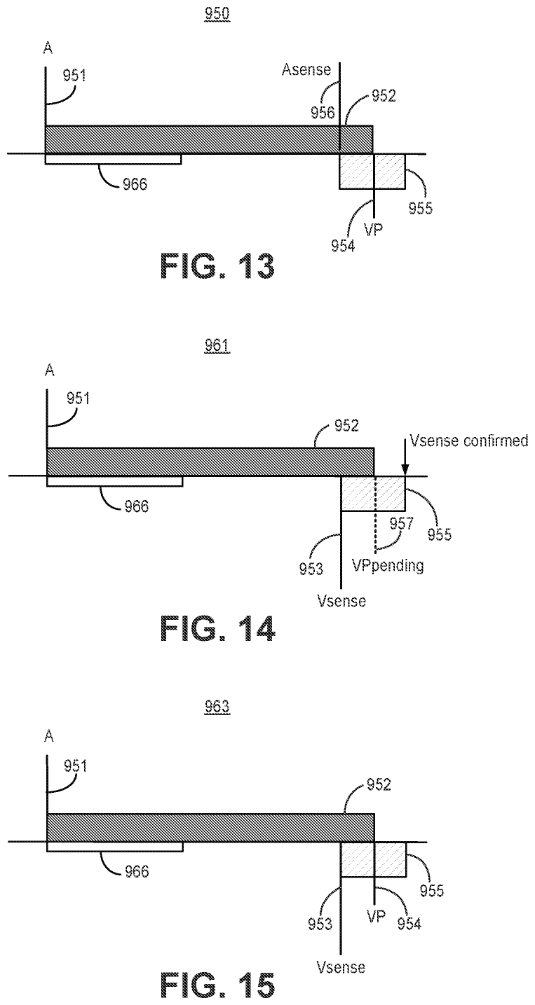

FIG. 13 is a diagram 950 of an Asense event signal 956 that triggers starting a confirmation delay 955 and a ventricular pacing interval 952 that expires during the confirmation delay 955. The ventricular pacing interval is an AV pacing interval in this example that expires before a Vsense event signal is received by control circuit 206. In this case, the atrial event (A) 951 may be a delivered atrial pacing pulse. In other instances, atrial event 951 may be a confirmed Asense event signal. Control circuit 206 may start the AV pacing interval 952 in response to the atrial event 951. In response to the atrial event 951 being an atrial pacing pulse, control circuit 206 may start the polarization delay 966 as described above (see block 904 of FIG. 12) and start the AV pacing interval 952 to schedule a ventricular pacing pulse (VP) 954. The scheduled ventricular pacing pulse 954 may be delivered during the confirmation delay 955 upon expiration of the AV pacing interval 952 when a Vsense event signal has not been received prior to the expiration of the AV pacing interval 952.

Referring again to FIG. 12, if the pacing escape interval that expires during the confirmation delay at block 915 was started to schedule a pacing pulse in the first heart chamber (“yes” branch of block 916), control circuit 206 may control pulse generator 202 to withhold the pending pacing pulse until expiration of the confirmation window. For example, when an atrial pacing escape interval expires at block 915 and the first chamber sensed event signal received at block 906 is an Asense event signal, control circuit 206 may return to block 912 to wait for a second chamber (Vsense) event signal and/or for the confirmation delay to expire at block 914. If the Asense event signal is confirmed at block 922, the pending atrial pacing pulse may be cancelled at block 926. If the Asense event signal is determined to be a CCOS signal and the second, Vsense event signal is confirmed at block 924, the pending atrial pacing pulse may be delivered at block 926.

In other examples, if a first heart chamber (atrial or ventricular) pacing escape interval expires such that a pending pacing pulse is scheduled to be delivered during the confirmation delay following a first heart chamber sensed event signal from the same heart chamber (e.g., an atrial pacing escape interval expires during the confirmation delay started in response to an Asense signal or a ventricular pacing escape interval expires during the confirmation delay started in response to a Vsense signal), the scheduled pacing pulse may be cancelled. The confirmation delay may be terminated and the first chamber event signal received at block 906 may be responded to as a confirmed sensed event by control circuit 206.

Referring again to block 915 of FIG. 12, if an atrial pacing escape interval expires at block 915 during the confirmation delay and the first chamber sensed event signal received at block 906 is a Vsense event signal, the pending atrial (second chamber) pacing pulse may be delivered at block 928. The confirmation delay may be terminated at block 930. However, if a ventricular pacing escape interval expires at block 915 after a Vsense signal starts the confirmation delay, control circuit 206 may control pulse generator 202 to delay the ventricular pacing pulse until the confirmation delay is expired and the Vsense event signal is either confirmed or determined to be a CCOS event. If the Vsense event signal is confirmed at block 922, the pending ventricular pacing pulse may be cancelled by control circuit 206 at block 926. If an Asense event signal is received during the confirmation delay and is confirmed at block 924, the delayed pending ventricular pacing pulse may be delivered by pulse generator 202 at block 926.

FIG. 14 is a diagram 961 of an example of a pacing response that may be performed by control circuit 206 and pulse generator 202 when a sensed event signal is received from a sensing channel corresponding to a first heart chamber and a pending pacing pulse is scheduled during the subsequent confirmation delay in the same heart chamber. In this example, instead of delivering the pending ventricular pacing pulse 957 at the expiration of the AV pacing interval 952, control circuit 206 controls pulse generator 202 to delay the pending ventricular pacing pulse 957 when the AV pacing interval 952 expires during the confirmation delay 955 started in response to a Vsense event signal 953. Control circuit 206 may wait to determine if the Vsense event signal 953 is confirmed before delivering the pending ventricular pacing pulse 957. In this example, an Asense event signal is not received during the confirmation delay 955 such that control circuit 206 confirms the Vsense event signal 953 at the expiration of the confirmation delay 955. In response to a confirmed Vsense event signal, control circuit 206 cancels the delayed ventricular pacing pulse such that no ventricular pacing pulse is delivered due to the Vsense event signal 953 occurring prior to expiration of the AV pacing interval 952.

FIG. 15 is a diagram 963 of yet another example of a pacing response that may be performed by control circuit 206 and pulse generator 202 when a sensed event signal is received from a sensing channel and a pending pacing pulse is scheduled during the subsequent confirmation delay in the same heart chamber. In this example, control circuit 206 starts an AV pacing interval 952 in response to an atrial event 951, which may be an atrial pacing pulse, to schedule a ventricular pacing pulse 954. A Vsense event signal 953 is received by control circuit 206 after the polarization delay 966. Control circuit 206 starts the confirmation delay 955. In this example, if the AV pacing interval 952 expires during the confirmation delay 955 before an Asense event signal is received during the confirmation delay 955, pulse generator 202 may deliver the scheduled ventricular pacing pulse 954 in response to the AV pacing interval 952 expiring. The Vsense signal 953 may be a true R-wave or an oversensed P-wave. The ventricular pacing pulse 954 may capture and pace the ventricles if the Vsense event signal 953 is due to an oversensed P-wave. If the Vsense event signal 953 is due to a true R-wave, the delivered ventricular pacing pulse 954 may occur during the ventricular absolute refractory period and may not cause depolarization of the ventricles. In this case, ventricular pacing pulse 954 may be considered a safety pace. In this example, the confirmation window 955 may be terminated upon delivery of the ventricular pacing pulse 954.

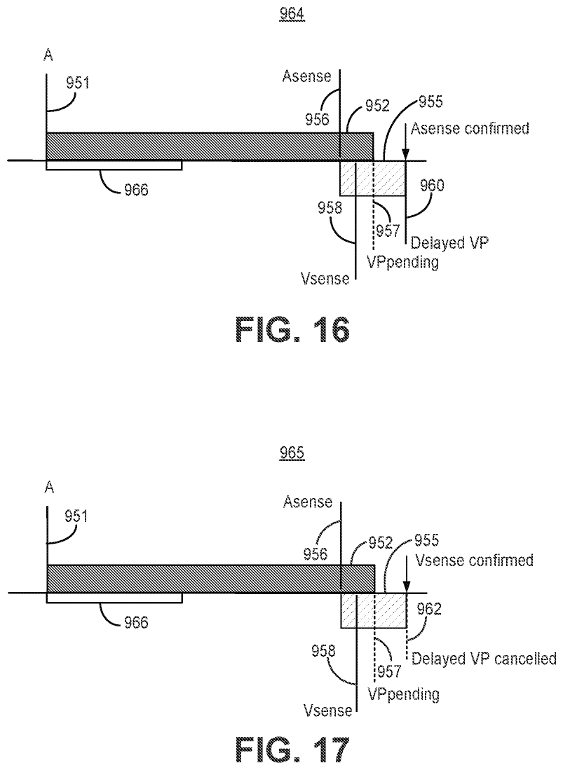

FIG. 16 is a diagram 964 of an Asense event signal 956 received by control circuit 206 followed by a Vsense event signal 958 received during the confirmation delay 955. The AV pacing interval 952 started in response to the atrial event 951 to schedule a pending ventricular pacing pulse 957 expires during the confirmation delay 955, in this case after the Vsense event signal 958 is received. In this example, control circuit 206 may control pulse generator 202 to delay the pending ventricular pacing pulse 957. When the Asense event signal 956 is confirmed at the expiration of the confirmation delay 955 based on the quantitative relationship determined from the atrial and ventricular EGM signal features, pulse generator 202 may deliver the delayed ventricular pacing pulse 960.

FIG. 17 is a diagram 965 of the same events shown in FIG. 16 except that, in this instance, the Vsense event signal 958 that is received during the confirmation delay 955, before the AV pacing interval 952 expires, is confirmed at the expiration of the confirmation delay 955 based on the quantitative relationship. Control circuit 206 may cancel the delayed ventricular pacing pulse 962 in response to the Vsense event signal 958 being confirmed. The delayed ventricular pacing pulse 962 is not delivered as indicated by dashed line and is appropriately inhibited due to the Vsense event signal 958, confirmed to be a true R-wave, received during the AV pacing interval 952.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device comprising:
sensing circuitry configured to sense a first cardiac signal and a second cardiac signal;
a control circuit configured to:
receive a first event signal associated with the first cardiac signal from the sensing circuitry;
receive a second event signal associated with the second cardiac signal from the sensing circuitry;
determine a quantitative relationship between a first feature of the first cardiac signal and a second feature of the second cardiac signal;
based on the quantitative relationship, confirm a sensed cardiac event signal as one of the first event signal or the second event signal; and
schedule a cardiac pacing pulse at one of:
a first pacing interval in response to confirming the sensed cardiac event signal as the first cardiac event signal; or
a second pacing interval different than the first pacing interval in response to confirming the sensed cardiac event signal as the second cardiac event signal; and
a pulse generator configured to deliver the scheduled cardiac pacing pulse.

2. (canceled)

3. (canceled)

4. The medical device of any of claim 1 wherein the sensing circuitry is further configured to:
sense a first cardiac event signal in response to the first cardiac signal crossing a first sensing threshold;
produce the first event signal received by the control circuit;
sense a second cardiac event signal in response to the second cardiac electrical signal crossing a second sensing threshold; and
produce the second event signal received by the control circuit.

5. (canceled)

6. The medical device of claim 1 wherein the control circuit is further configured to:
receive the first event signal as one of an atrial event signal or a ventricular event signal;
receive the second event signal as the other of the atrial event signal or the ventricular event signal;
when the first event signal is the atrial event signal:
compare the quantitative relationship to a P-wave confirmation threshold; and
confirm the sensed cardiac event signal based on the quantitative relationship by:
confirming the sensed cardiac event signal as being the atrial event signal in response to the quantitative relationship being less than the P-wave confirmation threshold; or
confirming the sensed cardiac event signal as being the ventricular event signal in response to the quantitative relationship being greater than the P-wave confirmation threshold; and
when the first event signal is the ventricular event signal:
compare the quantitative relationship to an R-wave confirmation threshold when the first event signal is the ventricular event signal; and
confirm the sensed cardiac event signal based on the quantitative relationship by:
confirming the sensed cardiac event signal as being the ventricular event signal in response to the quantitative relationship being greater than the R-wave confirmation threshold; or
confirming the sensed cardiac event signal as being the atrial event signal in response to the quantitative relationship being less than the R-wave confirmation threshold.

7. (canceled)

8. The medical device of claim 1 wherein the control circuit is further configured to confirm the sensed cardiac event signal based on the quantitative relationship by:
comparing the quantitative relationship to a P-wave confirmation threshold and to an R-wave confirmation threshold; and
confirming the sensed cardiac event signal as being one of:
an atrial event signal in response to the quantitative relationship being closer to the P-wave confirmation threshold than the R-wave confirmation threshold; or
a ventricular event signal in response to the quantitative relationship being closer to the R-wave confirmation threshold than to the P-wave confirmation threshold.

9. The medical device of claim 1 wherein the control circuit is further configured to determine the quantitative relationship by determining the first feature as a maximum peak amplitude of the first cardiac signal and the second feature as a maximum peak amplitude of the second cardiac signal.

10. The medical device of claim 1 wherein the control circuit is further configured to start one or more blanking period(s) and/or one or more refractory period(s) based on the confirmed sensed cardiac event signal.

11. The medical device of claim 1 wherein the control circuit is further configured to:
determine a first quantitative relationship of the first feature of the first cardiac signal and the second feature of the second cardiac signal in response to receiving the first event signal from the sensing circuitry without receiving the second event signal within a confirmation delay time from the first event signal;
determine a second quantitative relationship of the first feature of the first cardiac signal and the second feature of the second cardiac signal in response to receiving the second event signal from the sensing circuitry without receiving the first event signal within a confirmation delay time from the first event signal; and
maximize the difference between the first quantitative relationship and the second quantitative relationship by adjusting at least one of:
a first low pass filter cutoff frequency applied to the first cardiac signal by the sensing circuitry;
a first high pass filter cutoff frequency applied to the first cardiac signal by the sensing circuitry;
a second low pass filter cutoff frequency applied to the second cardiac signal by the sensing circuitry; and/or
a second high pass filter cutoff frequency applied to the second cardiac signal by the sensing circuitry.

12. (canceled)

13. The medical device of claim 1 wherein the control circuit is further configured to:
determine that the second cardiac event signal is received within a confirmation delay time from the first cardiac event signal; and
determine the quantitative relationship in response to the second cardiac event signal being received within the confirmation delay time.

14. The medical device of claim 13, wherein the control circuit is further configured to schedule the pacing pulse by
adjusting the one of the first pacing interval or the second pacing interval by the confirmation delay time.

15. The medical device of claim 1 wherein the control circuit is further configured to determine the quantitative relationship as one of:
a ratio of the first feature and the second feature;
a difference between the first feature and the second feature;
a sum of the first feature and the second feature; or
a product of the first feature and the second feature.

16. The medical device of claim 1 wherein the control circuit is further configured to:
start a pacing escape interval to schedule a pending pacing pulse;
start a confirmation delay in response to receiving the first event signal;
determine that the pacing escape interval expires during the confirmation delay;
delay the pending pacing pulse in response to the determining that the pacing escape interval expires during the confirmation delay;
and
control the pulse generator to deliver or withhold the delayed pending pacing pulse according to whether the sensed cardiac event signal is confirmed as the first event signal or the second event signal.

17. (canceled)

18. A method comprising:
sensing a first cardiac signal and a second cardiac signal;
receiving a first event signal associated with the first cardiac signal;
receiving a second event signal associated with the second cardiac signal;
determining a quantitative relationship between a first feature of the first cardiac signal and a second feature of the second cardiac signal;
based on the quantitative relationship, confirming a sensed cardiac event signal as being one of the first event signal or the second event signal;
scheduling a cardiac pacing pulse at one of:
a first pacing interval in response to confirming the sensed cardiac event signal as the first cardiac event signal; or
a second pacing interval different than the first pacing interval in response to confirming the sensed cardiac event signal as the second cardiac event signal; and
delivering the scheduled cardiac pacing pulse.

19. The method of claim 18 further comprising
receiving the first event signal as one of an atrial event signal or a ventricular event signal;
receiving the second event signal as the other of the atrial event signal or the ventricular event signal;
when the first event signal is the atrial event signal:
comparing the quantitative relationship to a P-wave confirmation threshold; and
confirming the sensed cardiac event signal based on the quantitative relationship by:
confirming the sensed cardiac event signal as being the atrial event signal in response to the quantitative relationship being less than the P-wave confirmation threshold; or
confirming the sensed cardiac event signal as being the ventricular event signal in response to the quantitative relationship being greater than the P-wave confirmation threshold; and
when the first event signal is the ventricular event signal:
comparing the quantitative relationship to an R-wave confirmation threshold when the first event signal is the ventricular event signal; and
confirming the sensed cardiac event signal based on the quantitative relationship by:
confirming the sensed cardiac event signal as being the ventricular event signal in response to the quantitative relationship being greater than the R-wave confirmation threshold; or
confirming the sensed cardiac event signal as being the atrial event signal in response to the quantitative relationship being less than the R-wave confirmation threshold.

20. The method of claim 18 wherein confirming the sensed cardiac event signal based on the quantitative relationship comprises:
comparing the quantitative relationship to a P-wave confirmation threshold and to an R-wave confirmation threshold; and
confirming the sensed cardiac event signal as being one of:
an atrial event signal in response to the quantitative relationship being closer to the P-wave confirmation threshold than the R-wave confirmation threshold; or
a ventricular event signal in response to the quantitative relationship being closer to the R-wave confirmation threshold than to the P-wave confirmation threshold.

21. The method of claim 18 further comprising determining the quantitative relationship by determining the first feature as a maximum peak amplitude of the first cardiac signal and the second feature as a maximum peak amplitude of the second cardiac signal.

22. The method of claim 18 further comprising:
determining a first quantitative relationship of the first feature of the first cardiac signal and the second feature of the second cardiac signal in response to receiving the first event signal without receiving the second event signal within a confirmation delay time from the first event signal;
determining a second quantitative relationship of the first feature of the first cardiac signal and the second feature of the second cardiac signal in response to receiving the second event signal without receiving the first event signal within a confirmation delay time from the first event signal; and
maximizing the difference between the first quantitative relationship and the second quantitative relationship by adjusting at least one of:
a first low pass filter cutoff frequency applied to the first cardiac signal;
a first high pass filter cutoff frequency applied to the first cardiac signal;
a second low pass filter cutoff frequency applied to the second cardiac signal; and/or
a second high pass filter cutoff frequency applied to the second cardiac signal.

23. The method of claim 18 further comprising:
determining that the second cardiac event signal is received within a confirmation delay time from the first cardiac event signal; and
determining the quantitative relationship in response to the second cardiac event signal being received within the confirmation delay time.

24. The method of claim 18 wherein determining the quantitative relationship comprises determining one of:
a ratio of the first feature and the second feature;
a difference between the first feature and the second feature;
a sum of the first feature and the second feature; or
a product of the first feature and the second feature.

25. The method of claim 18 further comprising:
starting a pacing escape interval to schedule a pending pacing pulse;
starting a confirmation delay in response to receiving the first event signal;
determining that the pacing escape interval expires during the confirmation delay;
delaying the pending pacing pulse in response to determining that the pacing escape interval expires during the confirmation delay; and
delivering or withholding the delayed pending pacing pulse according to whether the sensed cardiac event signal is confirmed as the first event signal or the second event signal.

26. A non-transitory computer readable medium storing a set of instructions that, when executed by a control circuit of a medical device, cause the medical device to:
sense a first event signal from a first cardiac signal;
sense a second event signal from a second cardiac signal;
determine a quantitative relationship between a first feature of the first cardiac signal and a second feature of the second cardiac signal;
based on the quantitative relationship, confirm a sensed cardiac event signal as being one of the first event signal or the second event signal;
schedule a cardiac pacing pulse at one of:
a first pacing interval in response to confirming the sensed cardiac event signal as the first cardiac event signal; or
a second pacing interval different than the first pacing interval in response to confirming the sensed cardiac event signal as the second cardiac event signal; and
deliver the scheduled cardiac pacing pulse.
